(19) United States
(12) Patent Application Publication
(10) Pub. No.: US 2021/0287308 A1
(43) Pub. Date: Sep. 16, 2021

(54) USING A WEARABLE APPARATUS IN SOCIAL EVENTS

(71) Applicant: OrCam Technologies Ltd., Jerusalem (IL)

(72) Inventors: Yonatan Wexler, Jeruslem (IL); Amnon Shashua, Mevaseret Zion (IL)

(73) Assignee: OrCam Technologies Ltd.

(21) Appl. No.: 17/336,874

(22) Filed: Jun. 2, 2021

Related U.S. Application Data
(63) Continuation of application No. PCT/IB2019/001304, filed on Dec. 12, 2019.
(60) Provisional application No. 62/778,936, filed on Dec. 13, 2018.
(60) Provisional application No. 62/780,970, filed on Dec. 18, 2018.
(60) Provisional application No. 62/790,042, filed on Jan. 9, 2019.

(51) Int. Cl. G06Q 50/00; G06F 1/16; G10L 15/08; G10L 15/22; G06T 7/70

(52) U.S. Cl. CPC G06Q 50/01; G06F 1/163; G10L 15/08; G10L 2015/088; G06T 7/70; G06T 2207/30196; G10L 15/22

(57) ABSTRACT

A hearing aid system may include an image sensor, an audio sensor, and at least one processor. The processor may be programmed to receive images captured by the image sensor from an environment of a user of a wearable apparatus; receive an audio signal representative of sound captured by the audio sensor from the environment of the user; determine, based on the images or the audio signal, whether an individual within the environment of the user is a recognized individual of the user; and subject to a determination the individual is not a recognized individual, identify the individual based on an external resource. The processor may further identify a content source associated with the individual; identify a first content item associated with the individual; and provide the first content item to a computing device associated with the user.

### CROSS REFERENCES TO RELATED APPLICATIONS

This application claims the benefit of priority of U.S. Provisional Patent Application No. 62/778,936, filed on Dec. 13, 2018; U.S. Provisional Patent Application No. 62/780,970, filed on Dec. 18, 2018; and U.S. Provisional Patent Application No. 62/790,042, filed on Jan. 9, 2019. All of the foregoing applications are incorporated herein by reference in their entirety.

### BACKGROUND

### Technical Field

This disclosure generally relates to devices and methods for capturing and processing images and audio from an environment of a user, and using information derived from captured images and audio.

### Background Information

Today, technological advancements make it possible for wearable devices to automatically capture images and audio, and store information that is associated with the captured images and audio. Certain devices have been used to digitally record aspects and personal experiences of one's life in an exercise typically called “lifelogging.” Some individuals log their life so they can retrieve moments from past activities, for example, social events, trips, etc. Lifelogging may also have significant benefits in other fields (e.g., business, fitness and healthcare, and social research). Lifelogging devices, while useful for tracking daily activities, may be improved with capability to enhance one's interaction in his environment with feedback and other advanced functionality based on the analysis of captured image and audio data.

Even though users can capture images and audio with their smartphones and some smartphone applications can process the captured information, smartphones may not be the best platform for serving as lifelogging apparatuses in view of their size and design. Lifelogging apparatuses should be small and light, so they can be easily worn. Moreover, with improvements in image capture devices, including wearable apparatuses, additional functionality may be provided to assist users in navigating in and around an environment, identifying persons and objects they encounter, and providing feedback to the users about their surroundings and activities. Therefore, there is a need for apparatuses and methods for automatically capturing and processing images and audio to provide useful information to users of the apparatuses, and for systems and methods to process and leverage information gathered by the apparatuses.

### SUMMARY

Embodiments consistent with the present disclosure provide devices and methods for automatically capturing and processing images and audio from an environment of a user, and systems and methods for processing information related to images and audio captured from the environment of the user.

In an exemplary embodiment, a wearable apparatus may comprise an image sensor configured to capture a plurality of images from the environment of a user of the wearable apparatus; an audio sensor configured to capture sound from the environment of the user; and at least one processor. The at least one processor may be programmed to receive the plurality of images captured by the image sensor; receive an audio signal representative of the sound captured by the audio sensor; determine, based on at least one of the plurality of images or the audio signal, whether an individual within the environment of the user is a recognized individual of the user; subject to a determination the individual is not a recognized individual, identify the individual based on an external resource; identify a content source associated with the individual; identify a first content item associated with the individual; and provide the first content item to a computing device associated with the user.

In another exemplary embodiment, a method for using a wearable apparatus in social events is disclosed. The method may comprise receiving a plurality of images captured from an environment of a user of a wearable apparatus by an image sensor; receiving an audio signal representative of a sound captured from the environment of the user by an audio sensor; determining, based on at least one of the plurality of images or the audio signal, whether an individual within the environment of the user is a recognized individual of the user; subject to a determination the individual is not a recognized individual, identifying the individual based on an external resource; identifying a content source associated with the individual; identifying a first content item associated with the individual; and providing the first content item to a computing device associated with the user.

In an exemplary embodiment, a wearable apparatus may comprise an image sensor configured to capture a plurality of images from an environment of a user of the wearable apparatus and at least one processor. The at least one processor may be programmed to: receive a first image depicting an individual associated with an order of a parcel; receive a second image captured by the image sensor, the second image depicting a recipient of the parcel; verify, based on the second image, whether the recipient is the individual depicted in the first image; and subject to a verification that the recipient is the individual depicted in the first image, store a delivery proof associated with the second image.

In another exemplary embodiment, a method for using a wearable apparatus for identification is disclosed. The method may comprise receiving a first image depicting an individual associated with an order of a parcel; receiving, from an image sensor configured to capture a plurality of images from an environment of a user of a wearable apparatus, a second image captured by the image sensor, the second image depicting a recipient of the parcel; verifying, based on the second image, whether the recipient is the individual depicted in the first image; and subject to a verification that the recipient is the individual depicted in the first image, storing a delivery proof associated with the second image.

In an embodiment, a wearable apparatus may comprise an image sensor configured to capture a first image from an environment of a user of the wearable apparatus. The wearable apparatus may also comprise at least one processor programmed to receive, from an external device, a second image and an identifying detail associated with the second image. The at least one processor may also be programmed to store the second image and the identifying detail in association with the second image and recognize a person depicted in the first image based on the second image and the identifying detail associated with the second image.

In an embodiment, a method may comprise capturing, by an image sensor of a wearable apparatus, a first image from an environment of a user of the wearable apparatus. The method may also comprise receiving, by at least one processor of the wearable apparatus, from an external device, a second image and an identifying detail associated with the second image. The method may further comprise storing, by the at least one processor the second image and the identifying detail in association with the second image. The method may also comprise recognizing, by the at least one processor, a person depicted in the first image based on the second image and the identifying detail associated with the second image.

Consistent with other disclosed embodiments, non-transitory computer-readable storage media may store program instructions, which are executed by at least one processor and perform any of the methods described herein.

The foregoing general description and the following detailed description are exemplary and explanatory only and are not restrictive of the claims.

### BRIEF DESCRIPTION OF THE DRAWINGS

The accompanying drawings, which are incorporated in and constitute a part of this disclosure, illustrate various disclosed embodiments. In the drawings:

FIG. 1A is a schematic illustration of an example of a user wearing a wearable apparatus according to a disclosed embodiment.

FIG. 1B is a schematic illustration of an example of the user wearing a wearable apparatus according to a disclosed embodiment.

FIG. 1C is a schematic illustration of an example of the user wearing a wearable apparatus according to a disclosed embodiment.

FIG. 1D is a schematic illustration of an example of the user wearing a wearable apparatus according to a disclosed embodiment.

FIG. 2 is a schematic illustration of an example system consistent with the disclosed embodiments.

FIG. 3A is a schematic illustration of an example of the wearable apparatus shown in FIG. 1A.

FIG. 3B is an exploded view of the example of the wearable apparatus shown in FIG. 3A.

FIG. 4A-4K are schematic illustrations of an example of the wearable apparatus shown in FIG. 1B from various viewpoints.

FIG. 5A is a block diagram illustrating an example of the components of a wearable apparatus according to a first embodiment.

FIG. 5B is a block diagram illustrating an example of the components of a wearable apparatus according to a second embodiment.

FIG. 5C is a block diagram illustrating an example of the components of a wearable apparatus according to a third embodiment.

FIG. 6 illustrates an exemplary embodiment of a memory containing software modules consistent with the present disclosure.

FIG. 7 is a schematic illustration of an embodiment of a wearable apparatus including an orientable image capture unit.

FIG. 8 is a schematic illustration of an embodiment of a wearable apparatus securable to an article of clothing consistent with the present disclosure.

FIG. 9 is a schematic illustration of a user wearing a wearable apparatus consistent with an embodiment of the present disclosure.

FIG. 10 is a schematic illustration of an embodiment of a wearable apparatus securable to an article of clothing consistent with the present disclosure.

FIG. 11 is a schematic illustration of an embodiment of a wearable apparatus securable to an article of clothing consistent with the present disclosure.

FIG. 12 is a schematic illustration of an embodiment of a wearable apparatus securable to an article of clothing consistent with the present disclosure.

FIG. 13 is a schematic illustration of an embodiment of a wearable apparatus securable to an article of clothing consistent with the present disclosure.

FIG. 14 is a schematic illustration of an embodiment of a wearable apparatus securable to an article of clothing consistent with the present disclosure.

FIG. 15 is a schematic illustration of an embodiment of a wearable apparatus power unit including a power source.

FIG. 16 is a schematic illustration of an exemplary embodiment of a wearable apparatus including protective circuitry.

FIG. 17A is a block diagram illustrating components of a wearable apparatus according to an example embodiment.

FIG. 17B is a block diagram illustrating the components of a wearable apparatus according to another example embodiment.

FIG. 17C is a block diagram illustrating the components of a wearable apparatus according to another example embodiment

FIG. 18A illustrates an example environment in which a user may interact with an individual consistent with the disclosed embodiments

FIG. 18B illustrates an example social media post that may be used to determine a content item consistent with the disclosed embodiments.

FIG. 19 is a flowchart showing an exemplary process for using a wearable apparatus in social events consistent with the disclosed embodiments.

FIG. 20 is a schematic illustration of an example system used for identification consistent with the disclosed embodiments.

FIG. 21A illustrates an example profile that may be stored in a database consistent with the disclosed embodiments.

FIG. 21B illustrates an example environment for delivery of a parcel consistent with the disclosed embodiments.

FIG. 22 is a flowchart showing an exemplary process for using a wearable apparatus for required identification consistent with the disclosed embodiments.

FIG. 23 is a schematic illustration of an example system consistent with the disclosed embodiments.

FIG. 24A is a schematic illustration of an environment of a user wearing a wearable apparatus according to a disclosed embodiment.

FIG. 24B is a schematic illustration of an example image of an environment of a user captured by a wearable apparatus according to a disclosed embodiment.

FIG. 24C is a schematic illustration of an example image according to a disclosed embodiment.

FIG. 25 is a flowchart of an exemplary process for recognizing a person in an image according to a disclosed embodiment.

### DETAILED DESCRIPTION

The following detailed description refers to the accompanying drawings. Wherever possible, the same reference numbers are used in the drawings and the following description to refer to the same or similar parts. While several illustrative embodiments are described herein, modifications, adaptations and other implementations are possible. For example, substitutions, additions or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope is defined by the appended claims.

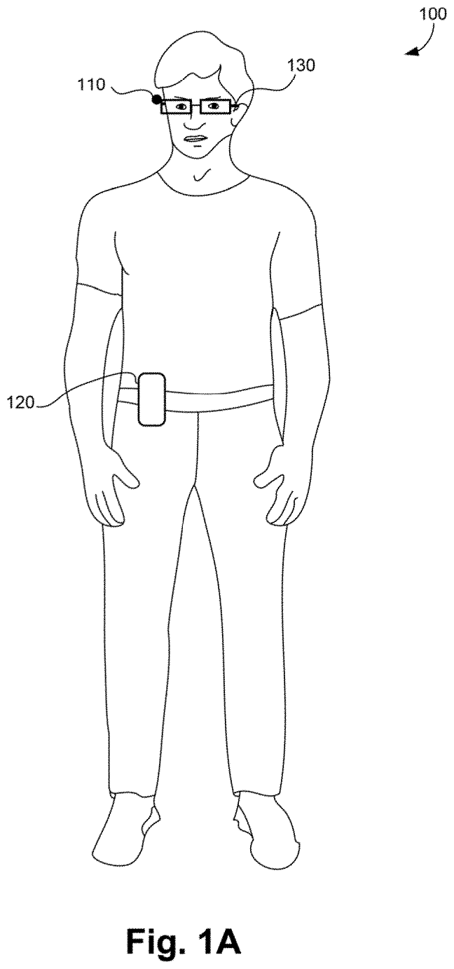

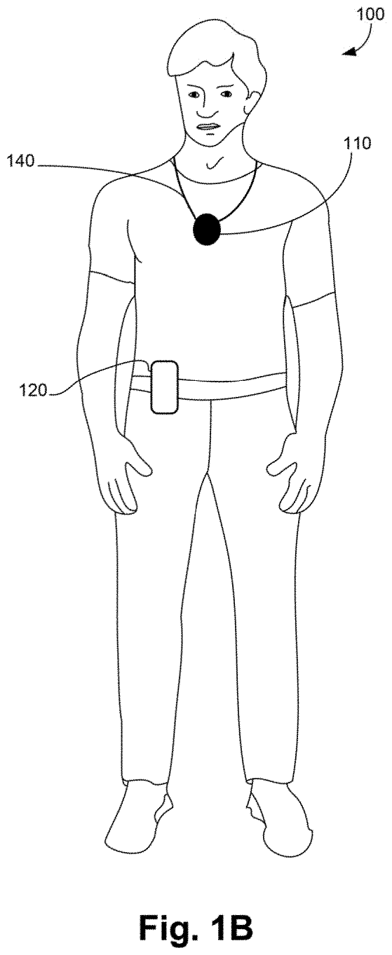

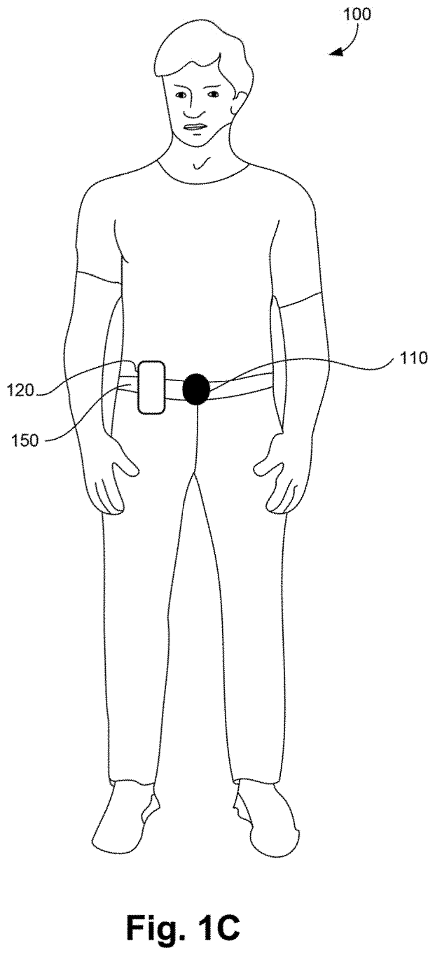

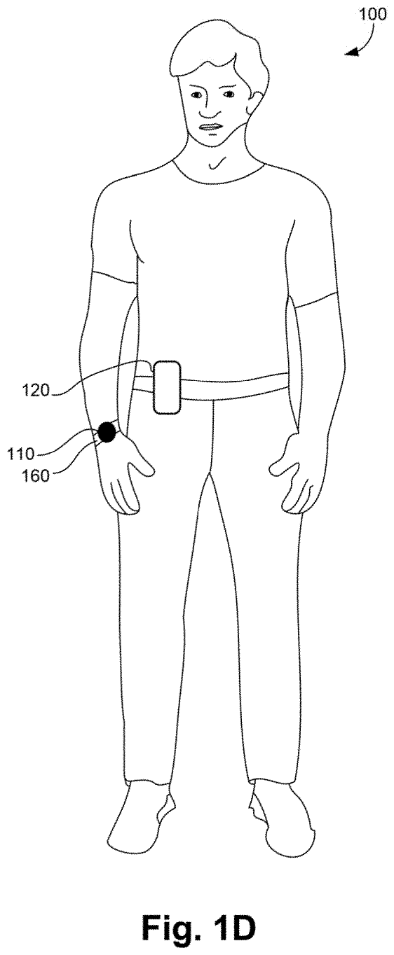

FIG. 1A illustrates a user 100 wearing an apparatus 110 that is physically connected (or integral) to glasses 130, consistent with the disclosed embodiments. Glasses 130 may be prescription glasses, magnifying glasses, non-prescription glasses, safety glasses, sunglasses, etc. Additionally, in some embodiments, glasses 130 may include parts of a frame and earpieces, nosepieces, etc., and one or no lenses. Thus, in some embodiments, glasses 130 may function primarily to support apparatus 110, and/or an augmented reality display device or other optical display device. In some embodiments, apparatus 110 may include an image sensor (not shown in FIG. 1A) for capturing real-time image data of the field-of-view of user 100. The term “image data” includes any form of data retrieved from optical signals in the near-infrared, infrared, visible, and ultraviolet spectrums. The image data may include video clips and/or photographs.

In some embodiments, apparatus 110 may communicate wirelessly or via a wire with a computing device 120. In some embodiments, computing device 120 may include, for example, a smartphone, or a tablet, or a dedicated processing unit, which may be portable (e.g., can be carried in a pocket of user 100). Although shown in FIG. 1A as an external device, in some embodiments, computing device 120 may be provided as part of wearable apparatus 110 or glasses 130, whether integral thereto or mounted thereon. In some embodiments, computing device 120 may be included in an augmented reality display device or optical head mounted display provided integrally or mounted to glasses 130. In other embodiments, computing device 120 may be provided as part of another wearable or portable apparatus of user 100 including a wrist-strap, a multifunctional watch, a button, a clip-on, etc. And in other embodiments, computing device 120 may be provided as part of another system, such as an on-board automobile computing or navigation system. A person skilled in the art can appreciate that different types of computing devices and arrangements of devices may implement the functionality of the disclosed embodiments. Accordingly, in other implementations, computing device 120 may include a Personal Computer (PC), laptop, an Internet server, etc.

FIG. 1B illustrates user 100 wearing apparatus 110 that is physically connected to a necklace 140, consistent with a disclosed embodiment. Such a configuration of apparatus 110 may be suitable for users that do not wear glasses some or all of the time. In this embodiment, user 100 can easily wear apparatus 110, and take it off.

FIG. 1C illustrates user 100 wearing apparatus 110 that is physically connected to a belt 150, consistent with a disclosed embodiment. Such a configuration of apparatus 110 may be designed as a belt buckle. Alternatively, apparatus 110 may include a clip for attaching to various clothing articles, such as belt 150, or a vest, a pocket, a collar, a cap or hat or other portion of a clothing article.

FIG. 1D illustrates user 100 wearing apparatus 110 that is physically connected to a wrist strap 160, consistent with a disclosed embodiment. Although the aiming direction of apparatus 110, according to this embodiment, may not match the field-of-view of user 100, apparatus 110 may include the ability to identify a hand-related trigger based on the tracked eye movement of a user 100 indicating that user 100 is looking in the direction of the wrist strap 160. Wrist strap 160 may also include an accelerometer, a gyroscope, or other sensor for determining movement or orientation of a user's 100 hand for identifying a hand-related trigger.

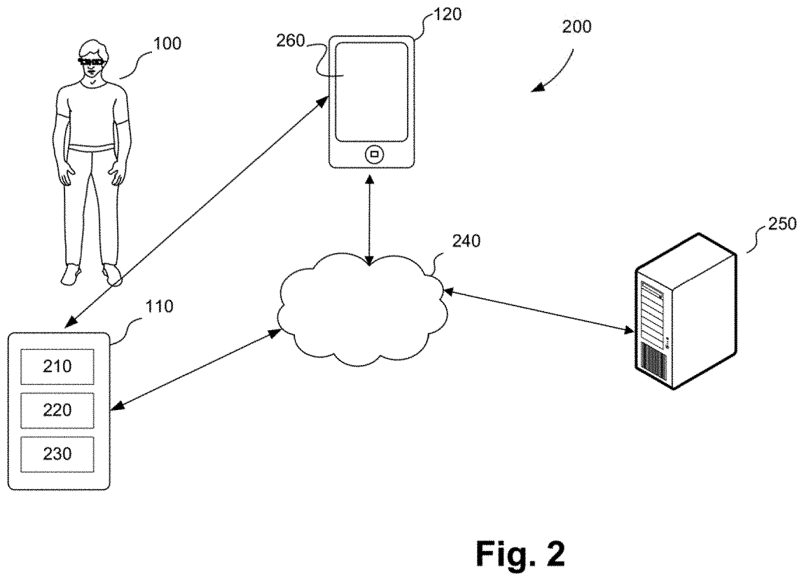

FIG. 2 is a schematic illustration of an exemplary system 200 including a wearable apparatus 110, worn by user 100, and an optional computing device 120 and/or a server 250 capable of communicating with apparatus 110 via a network 240, consistent with disclosed embodiments. In some embodiments, apparatus 110 may capture and analyze image data, identify a hand-related trigger present in the image data, and perform an action and/or provide feedback to a user 100, based at least in part on the identification of the hand-related trigger. In some embodiments, optional computing device 120 and/or server 250 may provide additional functionality to enhance interactions of user 100 with his or her environment, as described in greater detail below.

According to the disclosed embodiments, apparatus 110 may include an image sensor system 220 for capturing real-time image data of the field-of-view of user 100. In some embodiments, apparatus 110 may also include a processing unit 210 for controlling and performing the disclosed functionality of apparatus 110, such as to control the capture of image data, analyze the image data, and perform an action and/or output a feedback based on a hand-related trigger identified in the image data. According to the disclosed embodiments, a hand-related trigger may include a gesture performed by user 100 involving a portion of a hand of user 100. Further, consistent with some embodiments, a hand-related trigger may include a wrist-related trigger. Additionally, in some embodiments, apparatus 110 may include a feedback outputting unit 230 for producing an output of information to user 100.

As discussed above, apparatus 110 may include an image sensor 220 for capturing image data. The term “image sensor” refers to a device capable of detecting and converting optical signals in the near-infrared, infrared, visible, and ultraviolet spectrums into electrical signals. The electrical signals may be used to form an image or a video stream (i.e. image data) based on the detected signal. The term “image data” includes any form of data retrieved from optical signals in the near-infrared, infrared, visible, and ultraviolet spectrums. Examples of image sensors may include semiconductor charge-coupled devices (CCD), active pixel sensors in complementary metal-oxide-semiconductor (CMOS), or N-type metal-oxide-semiconductor (NMOS, Live MOS). In some cases, image sensor 220 may be part of a camera included in apparatus 110.

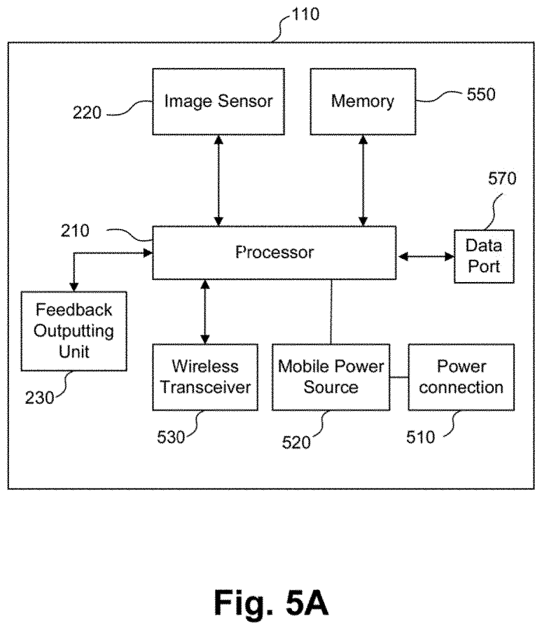

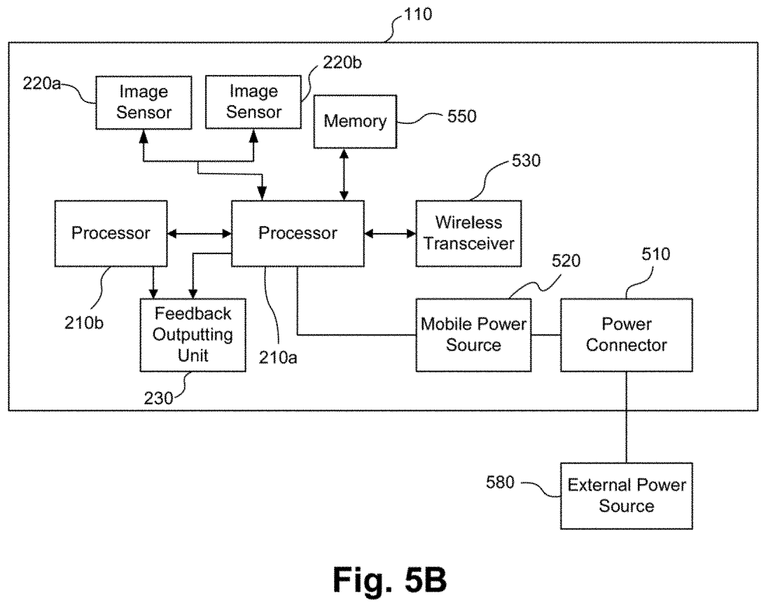

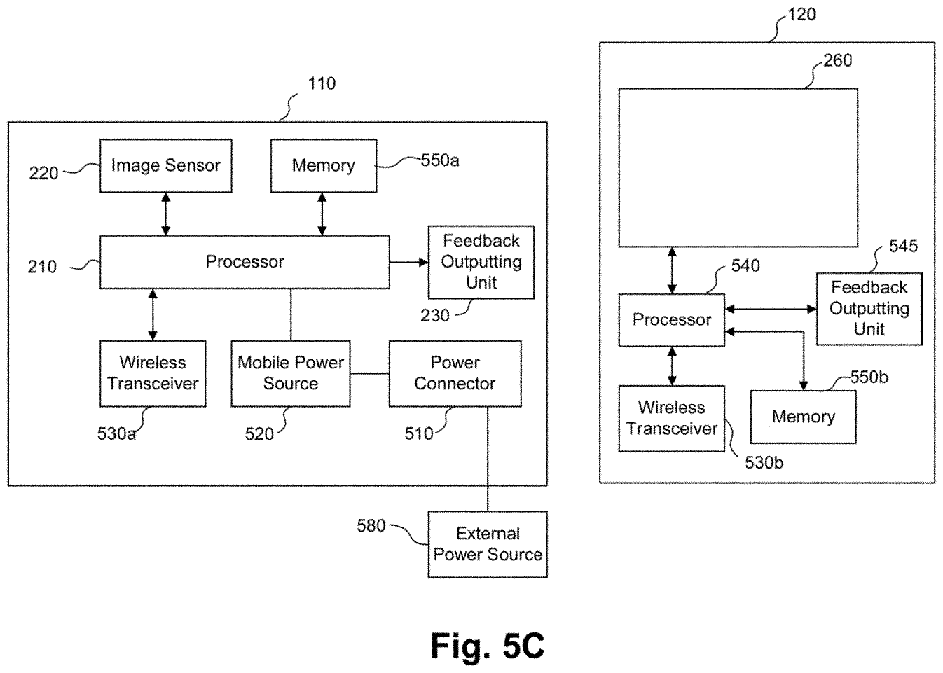

Apparatus 110 may also include a processor 210 for controlling image sensor 220 to capture image data and for analyzing the image data according to the disclosed embodiments. As discussed in further detail below with respect to FIG. 5A, processor 210 may include a “processing device” for performing logic operations on one or more inputs of image data and other data according to stored or accessible software instructions providing desired functionality. In some embodiments, processor 210 may also control feedback outputting unit 230 to provide feedback to user 100 including information based on the analyzed image data and the stored software instructions. As the term is used herein, a “processing device” may access memory where executable instructions are stored or, in some embodiments, a “processing device” itself may include executable instructions (e.g., stored in memory included in the processing device).

In some embodiments, the information or feedback information provided to user 100 may include time information. The time information may include any information related to a current time of day and, as described further below, may be presented in any sensory perceptive manner. In some embodiments, time information may include a current time of day in a preconfigured format (e.g., 2:30 pm or 14:30). Time information may include the time in the user's current time zone (e.g., based on a determined location of user 100), as well as an indication of the time zone and/or a time of day in another desired location. In some embodiments, time information may include a number of hours or minutes relative to one or more predetermined times of day. For example, in some embodiments, time information may include an indication that three hours and fifteen minutes remain until a particular hour (e.g., until 6:00 pm), or some other predetermined time. Time information may also include a duration of time passed since the beginning of a particular activity, such as the start of a meeting or the start of a jog, or any other activity. In some embodiments, the activity may be determined based on analyzed image data. In other embodiments, time information may also include additional information related to a current time and one or more other routine, periodic, or scheduled events. For example, time information may include an indication of the number of minutes remaining until the next scheduled event, as may be determined from a calendar function or other information retrieved from computing device 120 or server 250, as discussed in further detail below.

Feedback outputting unit 230 may include one or more feedback systems for providing the output of information to user 100. In the disclosed embodiments, the audible or visual feedback may be provided via any type of connected audible or visual system or both. Feedback of information according to the disclosed embodiments may include audible feedback to user 100 (e.g., using a Bluetooth™ or other wired or wirelessly connected speaker, or a bone conduction headphone). Feedback outputting unit 230 of some embodiments may additionally or alternatively produce a visible output of information to user 100, for example, as part of an augmented reality display projected onto a lens of glasses 130 or provided via a separate heads up display in communication with apparatus 110, such as a display 260 provided as part of computing device 120, which may include an onboard automobile heads up display, an augmented reality device, a virtual reality device, a smartphone, PC, table, etc.

The term “computing device” refers to a device including a processing unit and having computing capabilities. Some examples of computing device 120 include a PC, laptop, tablet, or other computing systems such as an on-board computing system of an automobile, for example, each configured to communicate directly with apparatus 110 or server 250 over network 240. Another example of computing device 120 includes a smartphone having a display 260. In some embodiments, computing device 120 may be a computing system configured particularly for apparatus 110, and may be provided integral to apparatus 110 or tethered thereto. Apparatus 110 can also connect to computing device 120 over network 240 via any known wireless standard (e.g., Wi-Fi, Bluetooth®, etc.), as well as near-filed capacitive coupling, and other short range wireless techniques, or via a wired connection. In an embodiment in which computing device 120 is a smartphone, computing device 120 may have a dedicated application installed therein. For example, user 100 may view on display 260 data (e.g., images, video clips, extracted information, feedback information, etc.) that originate from or are triggered by apparatus 110. In addition, user 100 may select part of the data for storage in server 250.

Network 240 may be a shared, public, or private network, may encompass a wide area or local area, and may be implemented through any suitable combination of wired and/or wireless communication networks. Network 240 may further comprise an intranet or the Internet. In some embodiments, network 240 may include short range or near-field wireless communication systems for enabling communication between apparatus 110 and computing device 120 provided in close proximity to each other, such as on or near a user's person, for example. Apparatus 110 may establish a connection to network 240 autonomously, for example, using a wireless module (e.g., Wi-Fi, cellular). In some embodiments, apparatus 110 may use the wireless module when being connected to an external power source, to prolong battery life. Further, communication between apparatus 110 and server 250 may be accomplished through any suitable communication channels, such as, for example, a telephone network, an extranet, an intranet, the Internet, satellite communications, off-line communications, wireless communications, transponder communications, a local area network (LAN), a wide area network (WAN), and a virtual private network (VPN).

As shown in FIG. 2, apparatus 110 may transfer or receive data to/from server 250 via network 240. In the disclosed embodiments, the data being received from server 250 and/or computing device 120 may include numerous different types of information based on the analyzed image data, including information related to a commercial product, or a person's identity, an identified landmark, and any other information capable of being stored in or accessed by server 250. In some embodiments, data may be received and transferred via computing device 120. Server 250 and/or computing device 120 may retrieve information from different data sources (e.g., a user specific database or a user's social network account or other account, the Internet, and other managed or accessible databases) and provide information to apparatus 110 related to the analyzed image data and a recognized trigger according to the disclosed embodiments. In some embodiments, calendar-related information retrieved from the different data sources may be analyzed to provide certain time information or a time-based context for providing certain information based on the analyzed image data.

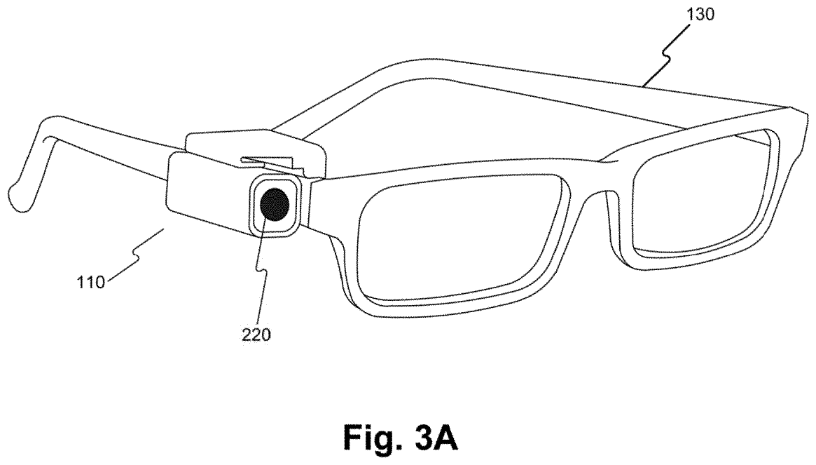

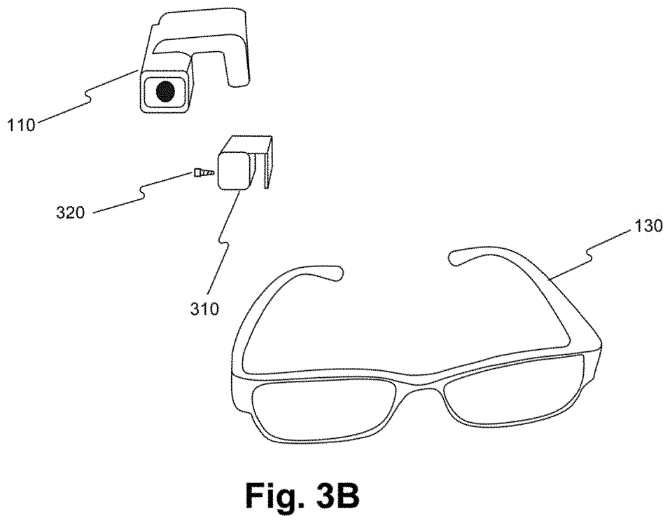

An example of wearable apparatus 110 incorporated with glasses 130 according to some embodiments (as discussed in connection with FIG. 1A) is shown in greater detail in FIG. 3A. In some embodiments, apparatus 110 may be associated with a structure (not shown in FIG. 3A) that enables easy detaching and reattaching of apparatus 110 to glasses 130. In some embodiments, when apparatus 110 attaches to glasses 130, image sensor 220 acquires a set aiming direction without the need for directional calibration. The set aiming direction of image sensor 220 may substantially coincide with the field-of-view of user 100. For example, a camera associated with image sensor 220 may be installed within apparatus 110 in a predetermined angle in a position facing slightly downwards (e.g., 5-15 degrees from the horizon). Accordingly, the set aiming direction of image sensor 220 may substantially match the field-of-view of user 100.

FIG. 3B is an exploded view of the components of the embodiment discussed regarding FIG. 3A. Attaching apparatus 110 to glasses 130 may take place in the following way. Initially, a support 310 may be mounted on glasses 130 using a screw 320, in the side of support 310. Then, apparatus 110 may be clipped on support 310 such that it is aligned with the field-of-view of user 100. The term “support” includes any device or structure that enables detaching and reattaching of a device including a camera to a pair of glasses or to another object (e.g., a helmet). Support 310 may be made from plastic (e.g., polycarbonate), metal (e.g., aluminum), or a combination of plastic and metal (e.g., carbon fiber graphite). Support 310 may be mounted on any kind of glasses (e.g., eyeglasses, sunglasses, 3D glasses, safety glasses, etc.) using screws, bolts, snaps, or any fastening means used in the art.

In some embodiments, support 310 may include a quick release mechanism for disengaging and reengaging apparatus 110. For example, support 310 and apparatus 110 may include magnetic elements. As an alternative example, support 310 may include a male latch member and apparatus 110 may include a female receptacle. In other embodiments, support 310 can be an integral part of a pair of glasses, or sold separately and installed by an optometrist. For example, support 310 may be configured for mounting on the arms of glasses 130 near the frame front, but before the hinge. Alternatively, support 310 may be configured for mounting on the bridge of glasses 130.

In some embodiments, apparatus 110 may be provided as part of a glasses frame 130, with or without lenses. Additionally, in some embodiments, apparatus 110 may be configured to provide an augmented reality display projected onto a lens of glasses 130 (if provided), or alternatively, may include a display for projecting time information, for example, according to the disclosed embodiments. Apparatus 110 may include the additional display or alternatively, may be in communication with a separately provided display system that may or may not be attached to glasses 130.

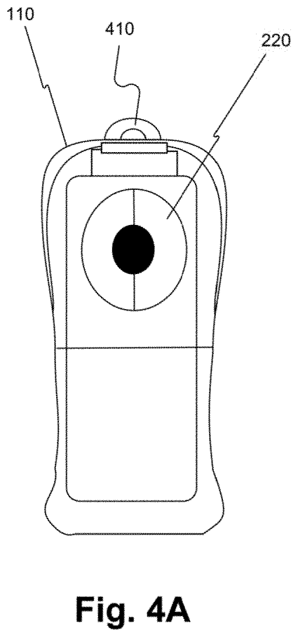

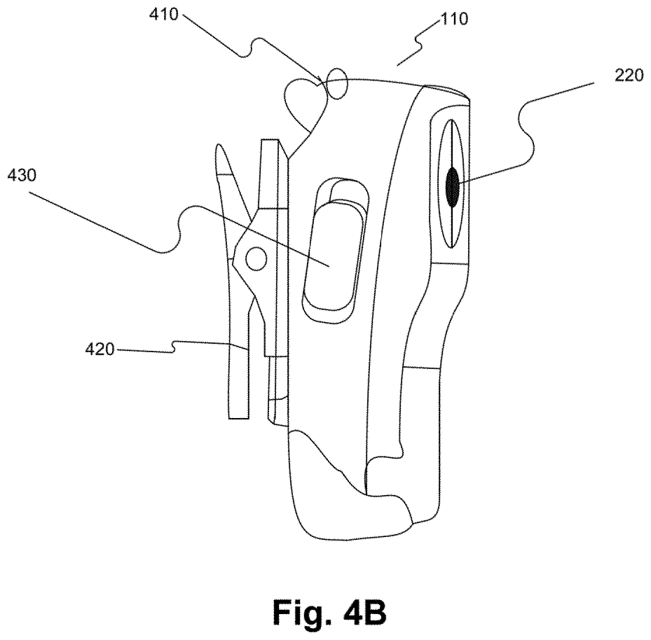

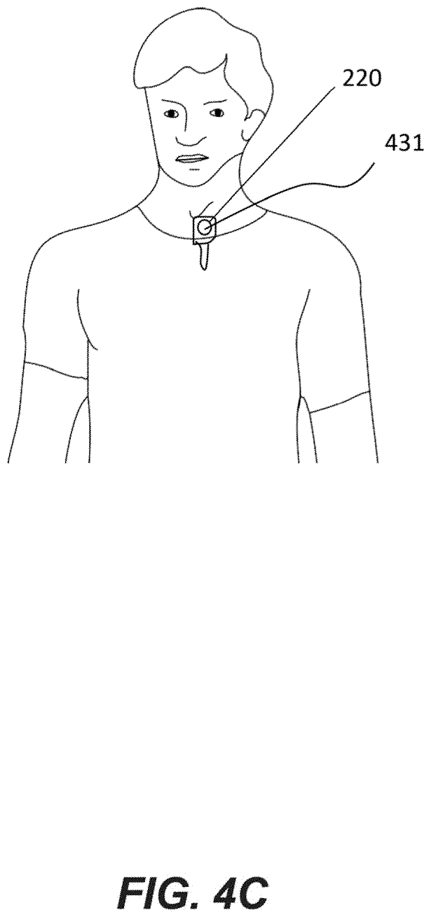

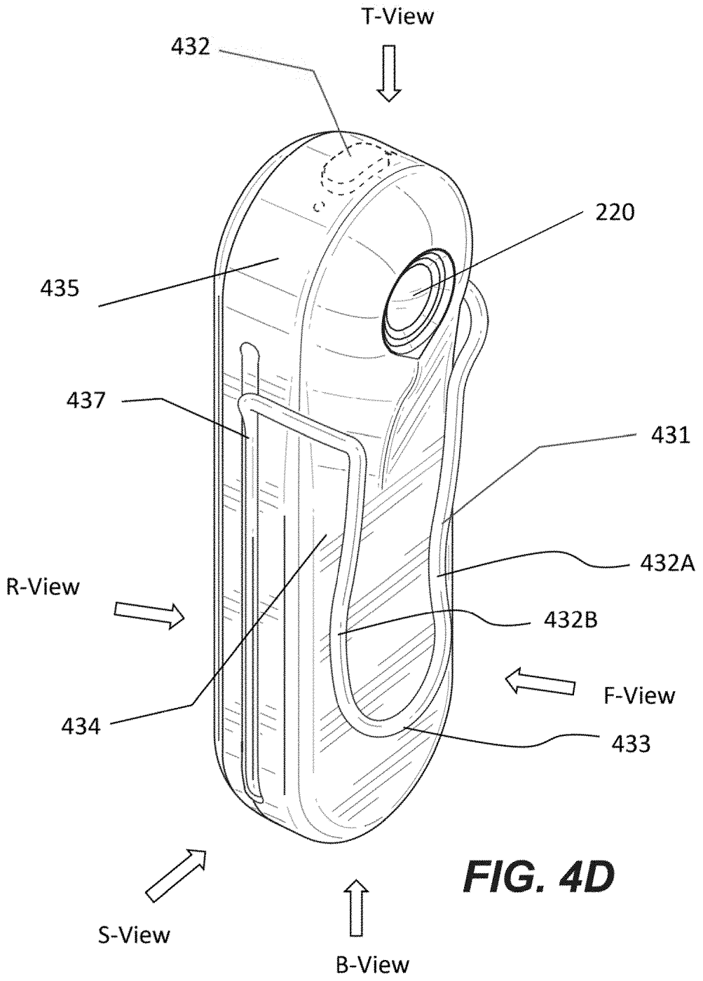

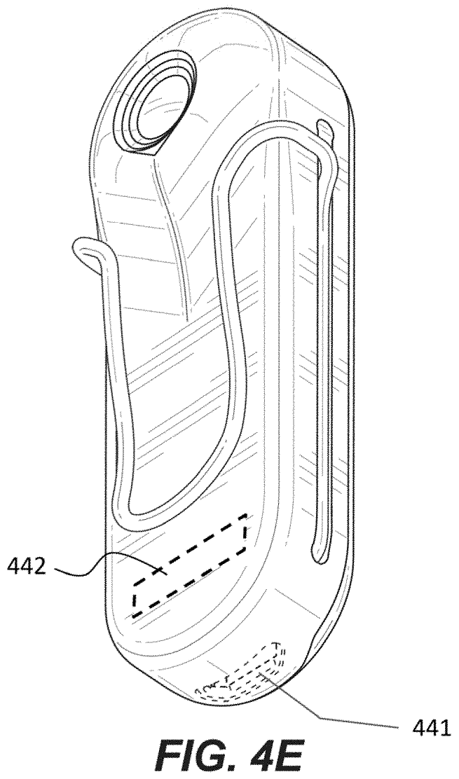

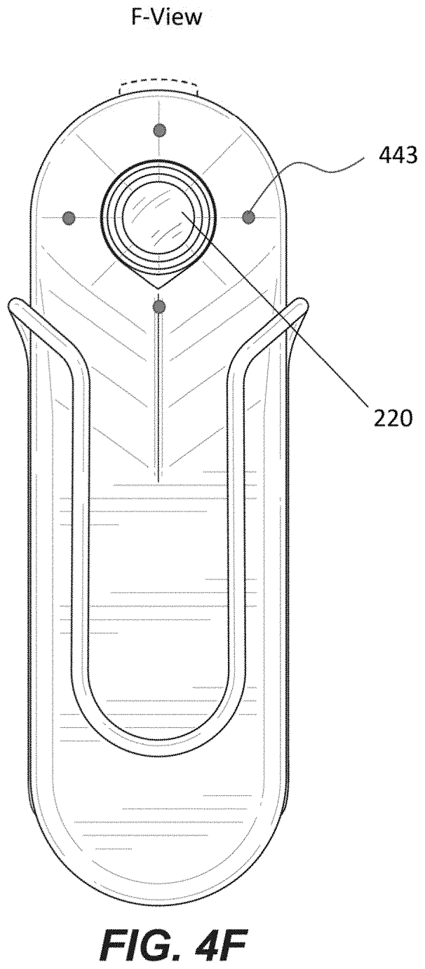

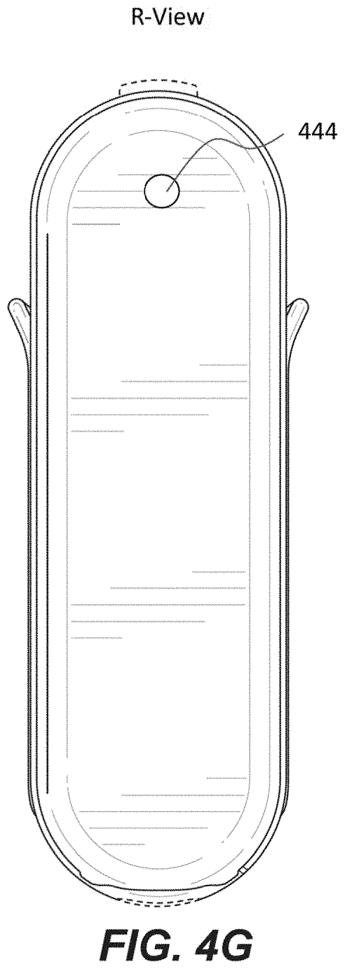

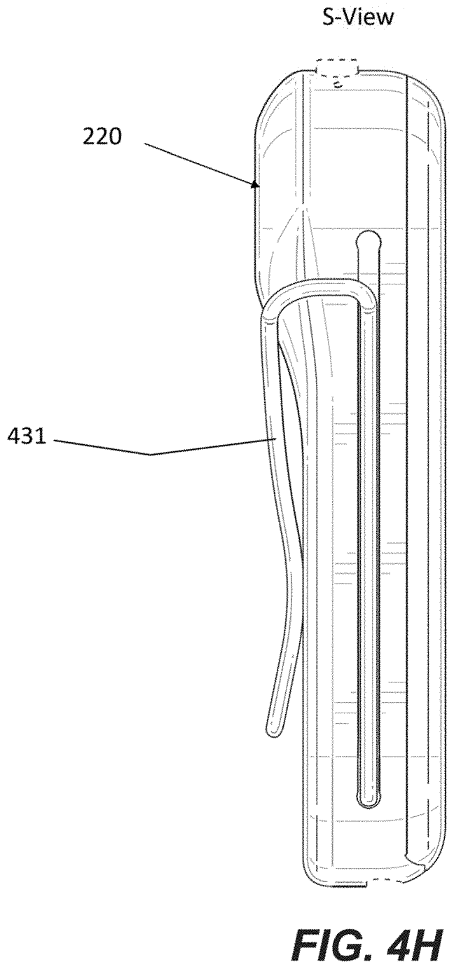

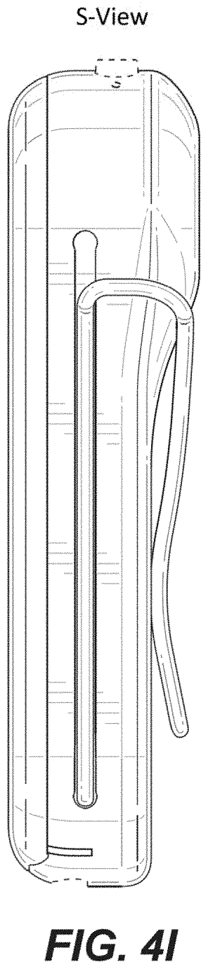

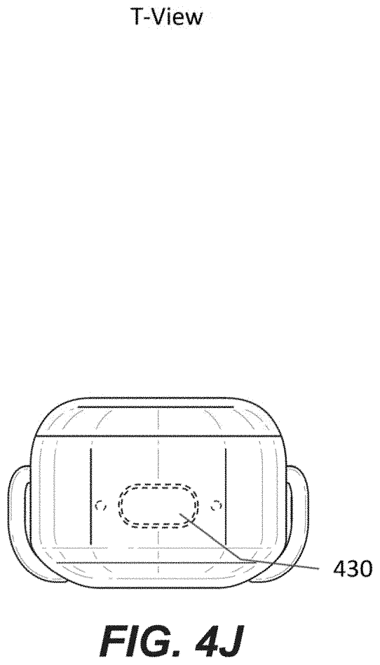

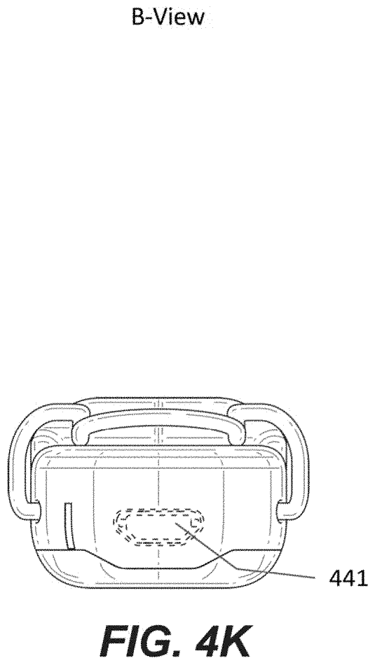

In some embodiments, apparatus 110 may be implemented in a form other than wearable glasses, as described above with respect to FIGS. 1B-1D, for example. FIG. 4A is a schematic illustration of an example of an additional embodiment of apparatus 110 from a front viewpoint of apparatus 110. Apparatus 110 includes an image sensor 220, a clip (not shown), a function button (not shown) and a hanging ring 410 for attaching apparatus 110 to, for example, necklace 140, as shown in FIG. 1B. When apparatus 110 hangs on necklace 140, the aiming direction of image sensor 220 may not fully coincide with the field-of-view of user 100, but the aiming direction would still correlate with the field-of-view of user 100.

FIG. 4B is a schematic illustration of the example of a second embodiment of apparatus 110, from a side orientation of apparatus 110. In addition to hanging ring 410, as shown in FIG. 4B, apparatus 110 may further include a clip 420. User 100 can use clip 420 to attach apparatus 110 to a shirt or belt 150, as illustrated in FIG. 1C. Clip 420 may provide an easy mechanism for disengaging and reengaging apparatus 110 from different articles of clothing. In other embodiments, apparatus 110 may include a female receptacle for connecting with a male latch of a car mount or universal stand.

In some embodiments, apparatus 110 includes a function button 430 for enabling user 100 to provide input to apparatus 110. Function button 430 may accept different types of tactile input (e.g., a tap, a click, a double-click, a long press, a right-to-left slide, a left-to-right slide). In some embodiments, each type of input may be associated with a different action. For example, a tap may be associated with the function of taking a picture, while a right-to-left slide may be associated with the function of recording a video.

Apparatus 110 may be attached to an article of clothing (e.g., a shirt, a belt, pants, etc.), of user 100 at an edge of the clothing using a clip 431 as shown in FIG. 4C. For example, the body of apparatus 100 may reside adjacent to the inside surface of the clothing with clip 431 engaging with the outside surface of the clothing. In such an embodiment, as shown in FIG. 4C, the image sensor 220 (e.g., a camera for visible light) may be protruding beyond the edge of the clothing. Alternatively, clip 431 may be engaging with the inside surface of the clothing with the body of apparatus 110 being adjacent to the outside of the clothing. In various embodiments, the clothing may be positioned between clip 431 and the body of apparatus 110.

An example embodiment of apparatus 110 is shown in FIG. 4D. Apparatus 110 includes clip 431 which may include points (e.g., 432A and 432B) in close proximity to a front surface 434 of a body 435 of apparatus 110. In an example embodiment, the distance between points 432A, 4328 and front surface 434 may be less than a typical thickness of a fabric of the clothing of user 100. For example, the distance between points 432A, 432B and surface 434 may be less than a thickness of a tee-shirt, e.g., less than a millimeter, less than 2 millimeters, less than 3 millimeters, etc., or, in some cases, points 432A, 432B of clip 431 may touch surface 434. In various embodiments, clip 431 may include a point 433 that does not touch surface 434, allowing the clothing to be inserted between clip 431 and surface 434.

FIG. 4D shows schematically different views of apparatus 110 defined as a front view (F-view), a rearview (R-view), a top view (T-view), a side view (S-view) and a bottom view (B-view). These views will be referred to when describing apparatus 110 in subsequent figures. FIG. 4D shows an example embodiment where clip 431 is positioned at the same side of apparatus 110 as sensor 220 (e.g., the front side of apparatus 110). Alternatively, clip 431 may be positioned at an opposite side of apparatus 110 as sensor 220 (e.g., the rear side of apparatus 110). In various embodiments, apparatus 110 may include function button 430, as shown in FIG. 4D.

Various views of apparatus 110 are illustrated in FIGS. 4E through 4K. For example, FIG. 4E shows a view of apparatus 110 with an electrical connection 441. Electrical connection 441 may be, for example, a USB port, that may be used to transfer data to/from apparatus 110 and provide electrical power to apparatus 110. In an example embodiment, connection 441 may be used to charge a battery 442 schematically shown in FIG. 4E. FIG. 4F shows F-view of apparatus 110, including sensor 220 and one or more microphones 443. In some embodiments, apparatus 110 may include several microphones 443 facing outwards, wherein microphones 443 are configured to obtain environmental sounds and sounds of various speakers communicating with user 100. FIG. 4G shows R-view of apparatus 110. In some embodiments, microphone 444 may be positioned at the rear side of apparatus 110, as shown in FIG. 4G. Microphone 444 may be used to detect an audio signal from user 100. It should be noted, that apparatus 110 may have microphones placed at any side (e.g., a front side, a rear side, a left side, a right side, a top side, or a bottom side) of apparatus 110. In various embodiments, some microphones may be at a first side (e.g., microphones 443 may be at the front of apparatus 110) and other microphones may be at a second side (e.g., microphone 444 may be at the back side of apparatus 110).

FIGS. 4H and 4I show different sides of apparatus 110 (i.e., S-view of apparatus 110) consisted with disclosed embodiments. For example, FIG. 4H shows the location of sensor 220 and an example shape of clip 431. FIG. 4J shows T-view of apparatus 110, including function button 430, and FIG. 4K shows B-view of apparatus 110 with electrical connection 441.

The example embodiments discussed above with respect to FIGS. 3A, 3B, 4A, and 4B are not limiting. In some embodiments, apparatus 110 may be implemented in any suitable configuration for performing the disclosed methods. For example, referring back to FIG. 2, the disclosed embodiments may implement an apparatus 110 according to any configuration including an image sensor 220 and a processor unit 210 to perform image analysis and for communicating with a feedback unit 230.

FIG. 5A is a block diagram illustrating the components of apparatus 110 according to an example embodiment. As shown in FIG. 5A, and as similarly discussed above, apparatus 110 includes an image sensor 220, a memory 550, a processor 210, a feedback outputting unit 230, a wireless transceiver 530, and a mobile power source 520. In other embodiments, apparatus 110 may also include buttons, other sensors such as a microphone, and inertial measurements devices such as accelerometers, gyroscopes, magnetometers, temperature sensors, color sensors, light sensors, etc. Apparatus 110 may further include a data port 570 and a power connection 510 with suitable interfaces for connecting with an external power source or an external device (not shown).

Processor 210, depicted in FIG. 5A, may include any suitable processing device. The term “processing device” includes any physical device having an electric circuit that performs a logic operation on input or inputs. For example, processing device may include one or more integrated circuits, microchips, microcontrollers, microprocessors, all or part of a central processing unit (CPU), graphics processing unit (GPU), digital signal processor (DSP), field-programmable gate array (FPGA), or other circuits suitable for executing instructions or performing logic operations. The instructions executed by the processing device may, for example, be pre-loaded into a memory integrated with or embedded into the processing device or may be stored in a separate memory (e.g., memory 550). Memory 550 may comprise a Random Access Memory (RAM), a Read-Only Memory (ROM), a hard disk, an optical disk, a magnetic medium, a flash memory, other permanent, fixed, or volatile memory, or any other mechanism capable of storing instructions.

Although, in the embodiment illustrated in FIG. 5A, apparatus 110 includes one processing device (e.g., processor 210), apparatus 110 may include more than one processing device. Each processing device may have a similar construction, or the processing devices may be of differing constructions that are electrically connected or disconnected from each other. For example, the processing devices may be separate circuits or integrated in a single circuit. When more than one processing device is used, the processing devices may be configured to operate independently or collaboratively. The processing devices may be coupled electrically, magnetically, optically, acoustically, mechanically or by other means that permit them to interact.

In some embodiments, processor 210 may process a plurality of images captured from the environment of user 100 to determine different parameters related to capturing subsequent images. For example, processor 210 can determine, based on information derived from captured image data, a value for at least one of the following: an image resolution, a compression ratio, a cropping parameter, frame rate, a focus point, an exposure time, an aperture size, and a light sensitivity. The determined value may be used in capturing at least one subsequent image. Additionally, processor 210 can detect images including at least one hand-related trigger in the environment of the user and perform an action and/or provide an output of information to a user via feedback outputting unit 230.

In another embodiment, processor 210 can change the aiming direction of image sensor 220. For example, when apparatus 110 is attached with clip 420, the aiming direction of image sensor 220 may not coincide with the field-of-view of user 100. Processor 210 may recognize certain situations from the analyzed image data and adjust the aiming direction of image sensor 220 to capture relevant image data. For example, in one embodiment, processor 210 may detect an interaction with another individual and sense that the individual is not fully in view, because image sensor 220 is tilted down. Responsive thereto, processor 210 may adjust the aiming direction of image sensor 220 to capture image data of the individual. Other scenarios are also contemplated where processor 210 may recognize the need to adjust an aiming direction of image sensor 220.

In some embodiments, processor 210 may communicate data to feedback-outputting unit 230, which may include any device configured to provide information to a user 100. Feedback outputting unit 230 may be provided as part of apparatus 110 (as shown) or may be provided external to apparatus 110 and communicatively coupled thereto. Feedback-outputting unit 230 may be configured to output visual or nonvisual feedback based on signals received from processor 210, such as when processor 210 recognizes a hand-related trigger in the analyzed image data.

The term “feedback” refers to any output or information provided in response to processing at least one image in an environment. In some embodiments, as similarly described above, feedback may include an audible or visible indication of time information, detected text or numerals, the value of currency, a branded product, a person's identity, the identity of a landmark or other environmental situation or condition including the street names at an intersection or the color of a traffic light, etc., as well as other information associated with each of these. For example, in some embodiments, feedback may include additional information regarding the amount of currency still needed to complete a transaction, information regarding the identified person, historical information or times and prices of admission etc. of a detected landmark etc. In some embodiments, feedback may include an audible tone, a tactile response, and/or information previously recorded by user 100. Feedback-outputting unit 230 may comprise appropriate components for outputting acoustical and tactile feedback. For example, feedback-outputting unit 230 may comprise audio headphones, a hearing aid type device, a speaker, a bone conduction headphone, interfaces that provide tactile cues, vibrotactile stimulators, etc. In some embodiments, processor 210 may communicate signals with an external feedback outputting unit 230 via a wireless transceiver 530, a wired connection, or some other communication interface. In some embodiments, feedback outputting unit 230 may also include any suitable display device for visually displaying information to user 100.

As shown in FIG. 5A, apparatus 110 includes memory 550. Memory 550 may include one or more sets of instructions accessible to processor 210 to perform the disclosed methods, including instructions for recognizing a hand-related trigger in the image data. In some embodiments memory 550 may store image data (e.g., images, videos) captured from the environment of user 100. In addition, memory 550 may store information specific to user 100, such as image representations of known individuals, favorite products, personal items, and calendar or appointment information, etc. In some embodiments, processor 210 may determine, for example, which type of image data to store based on available storage space in memory 550. In another embodiment, processor 210 may extract information from the image data stored in memory 550.

As further shown in FIG. 5A, apparatus 110 includes mobile power source 520. The term “mobile power source” includes any device capable of providing electrical power, which can be easily carried by hand (e.g., mobile power source 520 may weigh less than a pound). The mobility of the power source enables user 100 to use apparatus 110 in a variety of situations. In some embodiments, mobile power source 520 may include one or more batteries (e.g., nickel-cadmium batteries, nickel-metal hydride batteries, and lithium-ion batteries) or any other type of electrical power supply. In other embodiments, mobile power source 520 may be rechargeable and contained within a casing that holds apparatus 110. In yet other embodiments, mobile power source 520 may include one or more energy harvesting devices for converting ambient energy into electrical energy (e.g., portable solar power units, human vibration units, etc.).

Mobile power source 520 may power one or more wireless transceivers (e.g., wireless transceiver 530 in FIG. 5A). The term “wireless transceiver” refers to any device configured to exchange transmissions over an air interface by use of radio frequency, infrared frequency, magnetic field, or electric field. Wireless transceiver 530 may use any known standard to transmit and/or receive data (e.g., Wi-Fi, Bluetooth®, Bluetooth Smart, 802.15.4, or ZigBee). In some embodiments, wireless transceiver 530 may transmit data (e.g., raw image data, processed image data, extracted information) from apparatus 110 to computing device 120 and/or server 250. Wireless transceiver 530 may also receive data from computing device 120 and/or server 250. In other embodiments, wireless transceiver 530 may transmit data and instructions to an external feedback outputting unit 230.

FIG. 5B is a block diagram illustrating the components of apparatus 110 according to another example embodiment. In some embodiments, apparatus 110 includes a first image sensor 220a, a second image sensor 220b, a memory 550, a first processor 210a, a second processor 210b, a feedback outputting unit 230, a wireless transceiver 530, a mobile power source 520, and a power connector 510. In the arrangement shown in FIG. 5B, each of the image sensors may provide images in a different image resolution, or face a different direction. Alternatively, each image sensor may be associated with a different camera (e.g., a wide angle camera, a narrow angle camera, an IR camera, etc.). In some embodiments, apparatus 110 can select which image sensor to use based on various factors. For example, processor 210a may determine, based on available storage space in memory 550, to capture subsequent images in a certain resolution.

Apparatus 110 may operate in a first processing-mode and in a second processing-mode, such that the first processing-mode may consume less power than the second processing-mode. For example, in the first processing-mode, apparatus 110 may capture images and process the captured images to make real-time decisions based on an identifying hand-related trigger, for example. In the second processing-mode, apparatus 110 may extract information from stored images in memory 550 and delete images from memory 550. In some embodiments, mobile power source 520 may provide more than fifteen hours of processing in the first processing-mode and about three hours of processing in the second processing-mode. Accordingly, different processing-modes may allow mobile power source 520 to produce sufficient power for powering apparatus 110 for various time periods (e.g., more than two hours, more than four hours, more than ten hours, etc.).

In some embodiments, apparatus 110 may use first processor 210a in the first processing-mode when powered by mobile power source 520, and second processor 210b in the second processing-mode when powered by external power source 580 that is connectable via power connector 510. In other embodiments, apparatus 110 may determine, based on predefined conditions, which processors or which processing modes to use. Apparatus 110 may operate in the second processing-mode even when apparatus 110 is not powered by external power source 580. For example, apparatus 110 may determine that it should operate in the second processing-mode when apparatus 110 is not powered by external power source 580, if the available storage space in memory 550 for storing new image data is lower than a predefined threshold.

Although one wireless transceiver is depicted in FIG. 5B, apparatus 110 may include more than one wireless transceiver (e.g., two wireless transceivers). In an arrangement with more than one wireless transceiver, each of the wireless transceivers may use a different standard to transmit and/or receive data. In some embodiments, a first wireless transceiver may communicate with server 250 or computing device 120 using a cellular standard (e.g., LTE or GSM), and a second wireless transceiver may communicate with server 250 or computing device 120 using a short-range standard (e.g., Wi-Fi or Bluetooth®). In some embodiments, apparatus 110 may use the first wireless transceiver when the wearable apparatus is powered by a mobile power source included in the wearable apparatus, and use the second wireless transceiver when the wearable apparatus is powered by an external power source.

FIG. 5C is a block diagram illustrating the components of apparatus 110 according to another example embodiment including computing device 120. In this embodiment, apparatus 110 includes an image sensor 220, a memory 550a, a first processor 210, a feedback-outputting unit 230, a wireless transceiver 530a, a mobile power source 520, and a power connector 510. As further shown in FIG. 5C, computing device 120 includes a processor 540, a feedback-outputting unit 545, a memory 550b, a wireless transceiver 530b, and a display 260. One example of computing device 120 is a smartphone or tablet having a dedicated application installed therein. In other embodiments, computing device 120 may include any configuration such as an on-board automobile computing system, a PC, a laptop, and any other system consistent with the disclosed embodiments. In this example, user 100 may view feedback output in response to identification of a hand-related trigger on display 260. Additionally, user 100 may view other data (e.g., images, video clips, object information, schedule information, extracted information, etc.) on display 260. In addition, user 100 may communicate with server 250 via computing device 120.

In some embodiments, processor 210 and processor 540 are configured to extract information from captured image data. The term “extracting information” includes any process by which information associated with objects, individuals, locations, events, etc., is identified in the captured image data by any means known to those of ordinary skill in the art. In some embodiments, apparatus 110 may use the extracted information to send feedback or other real-time indications to feedback outputting unit 230 or to computing device 120. In some embodiments, processor 210 may identify in the image data the individual standing in front of user 100, and send computing device 120 the name of the individual and the last time user 100 met the individual. In another embodiment, processor 210 may identify in the image data, one or more visible triggers, including a hand-related trigger, and determine whether the trigger is associated with a person other than the user of the wearable apparatus to selectively determine whether to perform an action associated with the trigger. One such action may be to provide a feedback to user 100 via feedback-outputting unit 230 provided as part of (or in communication with) apparatus 110 or via a feedback unit 545 provided as part of computing device 120. For example, feedback-outputting unit 545 may be in communication with display 260 to cause the display 260 to visibly output information. In some embodiments, processor 210 may identify in the image data a hand-related trigger and send computing device 120 an indication of the trigger. Processor 540 may then process the received trigger information and provide an output via feedback outputting unit 545 or display 260 based on the hand-related trigger. In other embodiments, processor 540 may determine a hand-related trigger and provide suitable feedback similar to the above, based on image data received from apparatus 110. In some embodiments, processor 540 may provide instructions or other information, such as environmental information to apparatus 110 based on an identified hand-related trigger.

In some embodiments, processor 210 may identify other environmental information in the analyzed images, such as an individual standing in front user 100, and send computing device 120 information related to the analyzed information such as the name of the individual and the last time user 100 met the individual. In a different embodiment, processor 540 may extract statistical information from captured image data and forward the statistical information to server 250. For example, certain information regarding the types of items a user purchases, or the frequency a user patronizes a particular merchant, etc. may be determined by processor 540. Based on this information, server 250 may send computing device 120 coupons and discounts associated with the user's preferences.

When apparatus 110 is connected or wirelessly connected to computing device 120, apparatus 110 may transmit at least part of the image data stored in memory 550a for storage in memory 550b. In some embodiments, after computing device 120 confirms that transferring the part of image data was successful, processor 540 may delete the part of the image data. The term “delete” means that the image is marked as ‘deleted’ and other image data may be stored instead of it, but does not necessarily mean that the image data was physically removed from the memory.

As will be appreciated by a person skilled in the art having the benefit of this disclosure, numerous variations and/or modifications may be made to the disclosed embodiments. Not all components are essential for the operation of apparatus 110. Any component may be located in any appropriate apparatus and the components may be rearranged into a variety of configurations while providing the functionality of the disclosed embodiments. For example, in some embodiments, apparatus 110 may include a camera, a processor, and a wireless transceiver for sending data to another device. Therefore, the foregoing configurations are examples and, regardless of the configurations discussed above, apparatus 110 can capture, store, and/or process images.

Further, the foregoing and following description refers to storing and/or processing images or image data. In the embodiments disclosed herein, the stored and/or processed images or image data may comprise a representation of one or more images captured by image sensor 220. As the term is used herein, a “representation” of an image (or image data) may include an entire image or a portion of an image. A representation of an image (or image data) may have the same resolution or a lower resolution as the image (or image data), and/or a representation of an image (or image data) may be altered in some respect (e.g., be compressed, have a lower resolution, have one or more colors that are altered, etc.).

For example, apparatus 110 may capture an image and store a representation of the image that is compressed as a .JPG file. As another example, apparatus 110 may capture an image in color, but store a black-and-white representation of the color image. As yet another example, apparatus 110 may capture an image and store a different representation of the image (e.g., a portion of the image). For example, apparatus 110 may store a portion of an image that includes a face of a person who appears in the image, but that does not substantially include the environment surrounding the person. Similarly, apparatus 110 may, for example, store a portion of an image that includes a product that appears in the image, but does not substantially include the environment surrounding the product. As yet another example, apparatus 110 may store a representation of an image at a reduced resolution (i.e., at a resolution that is of a lower value than that of the captured image). Storing representations of images may allow apparatus 110 to save storage space in memory 550. Furthermore, processing representations of images may allow apparatus 110 to improve processing efficiency and/or help to preserve battery life.

In addition to the above, in some embodiments, any one of apparatus 110 or computing device 120, via processor 210 or 540, may further process the captured image data to provide additional functionality to recognize objects and/or gestures and/or other information in the captured image data. In some embodiments, actions may be taken based on the identified objects, gestures, or other information. In some embodiments, processor 210 or 540 may identify in the image data, one or more visible triggers, including a hand-related trigger, and determine whether the trigger is associated with a person other than the user to determine whether to perform an action associated with the trigger.

Some embodiments of the present disclosure may include an apparatus securable to an article of clothing of a user. Such an apparatus may include two portions, connectable by a connector. A capturing unit may be designed to be worn on the outside of a user's clothing, and may include an image sensor for capturing images of a user's environment. The capturing unit may be connected to or connectable to a power unit, which may be configured to house a power source and a processing device. The capturing unit may be a small device including a camera or other device for capturing images. The capturing unit may be designed to be inconspicuous and unobtrusive, and may be configured to communicate with a power unit concealed by a user's clothing. The power unit may include bulkier aspects of the system, such as transceiver antennas, at least one battery, a processing device, etc. In some embodiments, communication between the capturing unit and the power unit may be provided by a data cable included in the connector, while in other embodiments, communication may be wirelessly achieved between the capturing unit and the power unit. Some embodiments may permit alteration of the orientation of an image sensor of the capture unit, for example to better capture images of interest.

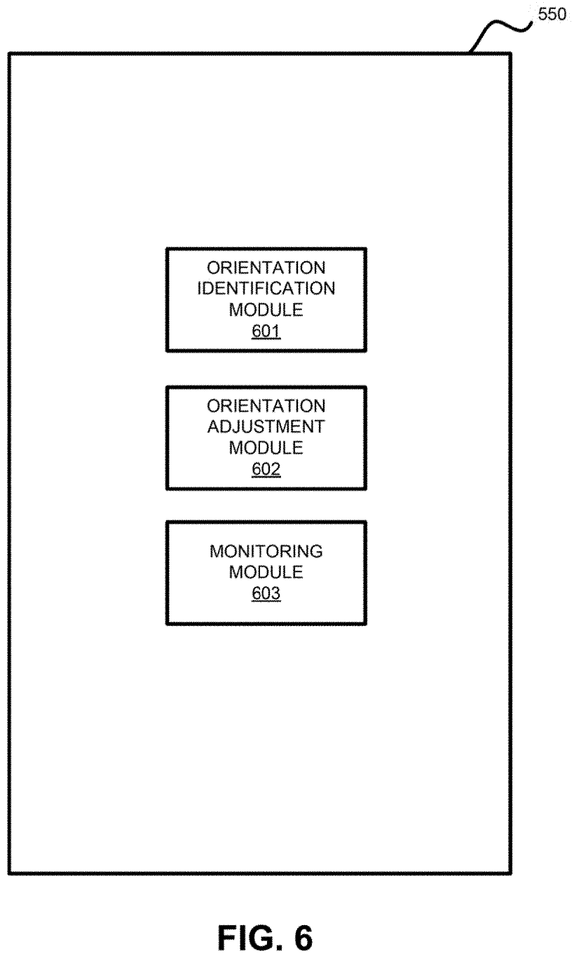

FIG. 6 illustrates an exemplary embodiment of a memory containing software modules consistent with the present disclosure. Included in memory 550 are orientation identification module 601, orientation adjustment module 602, and motion tracking module 603. Modules 601, 602, 603 may contain software instructions for execution by at least one processing device, e.g., processor 210, included with a wearable apparatus. Orientation identification module 601, orientation adjustment module 602, and motion tracking module 603 may cooperate to provide orientation adjustment for a capturing unit incorporated into wireless apparatus 110.

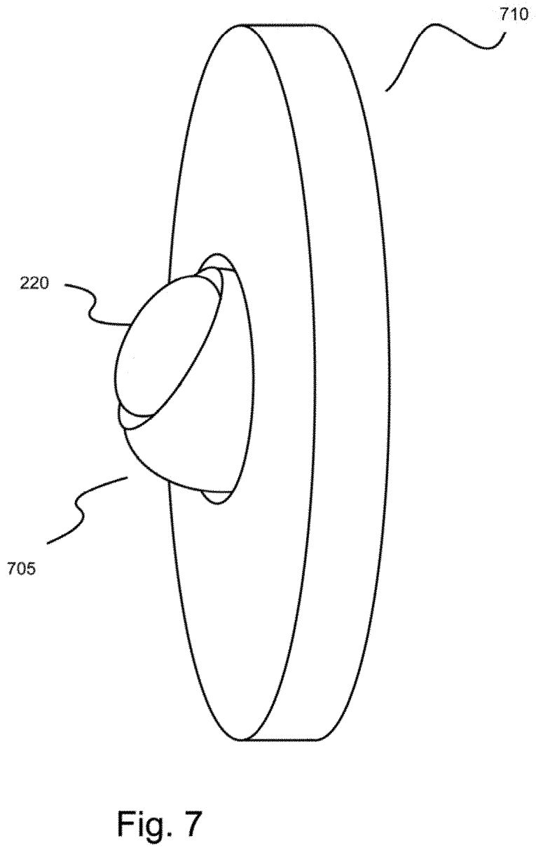

FIG. 7 illustrates an exemplary capturing unit 710 including an orientation adjustment unit 705. Orientation adjustment unit 705 may be configured to permit the adjustment of image sensor 220. As illustrated in FIG. 7, orientation adjustment unit 705 may include an eye-ball type adjustment mechanism. In alternative embodiments, orientation adjustment unit 705 may include gimbals, adjustable stalks, pivotable mounts, and any other suitable unit for adjusting an orientation of image sensor 220.

Image sensor 220 may be configured to be movable with the head of user 100 in such a manner that an aiming direction of image sensor 220 substantially coincides with a field of view of user 100. For example, as described above, a camera associated with image sensor 220 may be installed within capturing unit 710 at a predetermined angle in a position facing slightly upwards or downwards, depending on an intended location of capturing unit 710. Accordingly, the set aiming direction of image sensor 220 may match the field-of-view of user 100. In some embodiments, processor 210 may change the orientation of image sensor 220 using image data provided from image sensor 220. For example, processor 210 may recognize that a user is reading a book and determine that the aiming direction of image sensor 220 is offset from the text. That is, because the words in the beginning of each line of text are not fully in view, processor 210 may determine that image sensor 220 is tilted in the wrong direction. Responsive thereto, processor 210 may adjust the aiming direction of image sensor 220.

Orientation identification module 601 may be configured to identify an orientation of an image sensor 220 of capturing unit 710. An orientation of an image sensor 220 may be identified, for example, by analysis of images captured by image sensor 220 of capturing unit 710, by tilt or attitude sensing devices within capturing unit 710, and by measuring a relative direction of orientation adjustment unit 705 with respect to the remainder of capturing unit 710.

Orientation adjustment module 602 may be configured to adjust an orientation of image sensor 220 of capturing unit 710. As discussed above, image sensor 220 may be mounted on an orientation adjustment unit 705 configured for movement. Orientation adjustment unit 705 may be configured for rotational and/or lateral movement in response to commands from orientation adjustment module 602. In some embodiments orientation adjustment unit 705 may be adjust an orientation of image sensor 220 via motors, electromagnets, permanent magnets, and/or any suitable combination thereof.

In some embodiments, monitoring module 603 may be provided for continuous monitoring. Such continuous monitoring may include tracking a movement of at least a portion of an object included in one or more images captured by the image sensor. For example, in one embodiment, apparatus 110 may track an object as long as the object remains substantially within the field-of-view of image sensor 220. In additional embodiments, monitoring module 603 may engage orientation adjustment module 602 to instruct orientation adjustment unit 705 to continually orient image sensor 220 towards an object of interest. For example, in one embodiment, monitoring module 603 may cause image sensor 220 to adjust an orientation to ensure that a certain designated object, for example, the face of a particular person, remains within the field-of view of image sensor 220, even as that designated object moves about. In another embodiment, monitoring module 603 may continuously monitor an area of interest included in one or more images captured by the image sensor. For example, a user may be occupied by a certain task, for example, typing on a laptop, while image sensor 220 remains oriented in a particular direction and continuously monitors a portion of each image from a series of images to detect a trigger or other event. For example, image sensor 210 may be oriented towards a piece of laboratory equipment and monitoring module 603 may be configured to monitor a status light on the laboratory equipment for a change in status, while the user's attention is otherwise occupied.

In some embodiments consistent with the present disclosure, capturing unit 710 may include a plurality of image sensors 220. The plurality of image sensors 220 may each be configured to capture different image data. For example, when a plurality of image sensors 220 are provided, the image sensors 220 may capture images having different resolutions, may capture wider or narrower fields of view, and may have different levels of magnification. Image sensors 220 may be provided with varying lenses to permit these different configurations. In some embodiments, a plurality of image sensors 220 may include image sensors 220 having different orientations. Thus, each of the plurality of image sensors 220 may be pointed in a different direction to capture different images. The fields of view of image sensors 220 may be overlapping in some embodiments. The plurality of image sensors 220 may each be configured for orientation adjustment, for example, by being paired with an image adjustment unit 705. In some embodiments, monitoring module 603, or another module associated with memory 550, may be configured to individually adjust the orientations of the plurality of image sensors 220 as well as to turn each of the plurality of image sensors 220 on or off as may be required or preferred. In some embodiments, monitoring an object or person captured by an image sensor 220 may include tracking movement of the object across the fields of view of the plurality of image sensors 220.

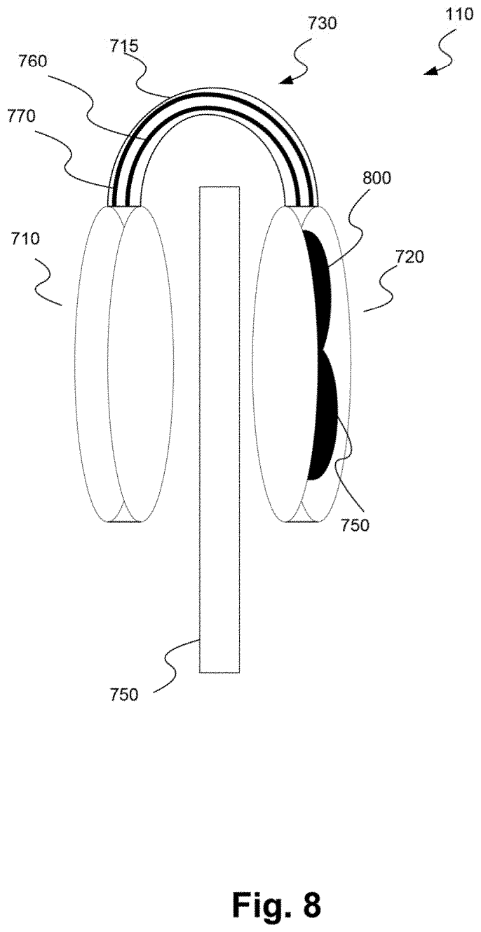

Embodiments consistent with the present disclosure may include connectors configured to connect a capturing unit and a power unit of a wearable apparatus. Capturing units consistent with the present disclosure may include least one image sensor configured to capture images of an environment of a user. Power units consistent with the present disclosure may be configured to house a power source and/or at least one processing device. Connectors consistent with the present disclosure may be configured to connect the capturing unit and the power unit, and may be configured to secure the apparatus to an article of clothing such that the capturing unit is positioned over an outer surface of the article of clothing and the power unit is positioned under an inner surface of the article of clothing. Exemplary embodiments of capturing units, connectors, and power units consistent with the disclosure are discussed in further detail with respect to FIGS. 8-14.

FIG. 8 is a schematic illustration of an embodiment of wearable apparatus 110 securable to an article of clothing consistent with the present disclosure. As illustrated in FIG. 8, capturing unit 710 and power unit 720 may be connected by a connector 730 such that capturing unit 710 is positioned on one side of an article of clothing 750 and power unit 720 is positioned on the opposite side of the clothing 750. In some embodiments, capturing unit 710 may be positioned over an outer surface of the article of clothing 750 and power unit 720 may be located wider an inner surface of the article of clothing 750. The power unit 720 may be configured to be placed against the skin of a user.

Capturing unit 710 may include an image sensor 220 and an orientation adjustment unit 705 (as illustrated in FIG. 7). Power unit 720 may include mobile power source 520 and processor 210. Power unit 720 may further include any combination of elements previously discussed that may be a part of wearable apparatus 110, including, but not limited to, wireless transceiver 530, feedback outputting unit 230, memory 550, and data port 570.

Connector 730 may include a clip 715 or other mechanical connection designed to clip or attach capturing unit 710 and power unit 720 to an article of clothing 750 as illustrated in FIG. 8. As illustrated, clip 715 may connect to each of capturing unit 710 and power unit 720 at a perimeter thereof, and may wrap around an edge of the article of clothing 750 to affix the capturing unit 710 and power unit 720 in place. Connector 730 may further include a power cable 760 and a data cable 770. Power cable 760 may be capable of conveying power from mobile power source 520 to image sensor 220 of capturing unit 710. Power cable 760 may also be configured to provide power to any other elements of capturing unit 710, e.g., orientation adjustment unit 705. Data cable 770 may be capable of conveying captured image data from image sensor 220 in capturing unit 710 to processor 800 in the power unit 720. Data cable 770 may be further capable of conveying additional data between capturing unit 710 and processor 800, e.g., control instructions for orientation adjustment unit 705.

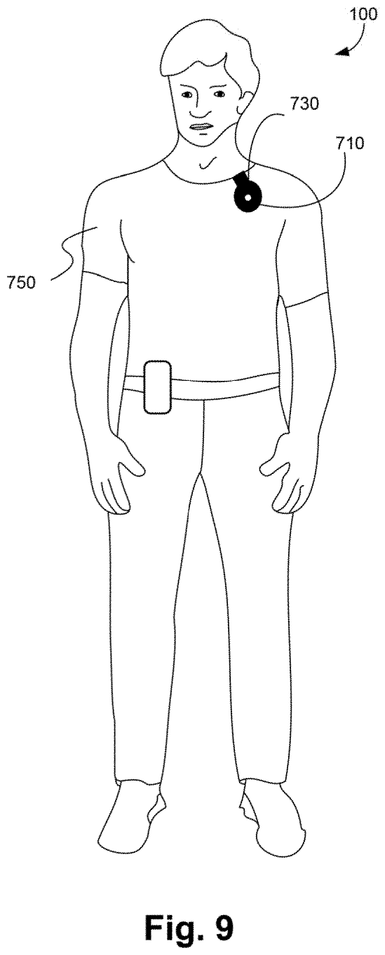

FIG. 9 is a schematic illustration of a user 100 wearing a wearable apparatus 110 consistent with an embodiment of the present disclosure. As illustrated in FIG. 9, capturing unit 710 is located on an exterior surface of the clothing 750 of user 100. Capturing unit 710 is connected to power unit 720 (not seen in this illustration) via connector 730, which wraps around an edge of clothing 750.

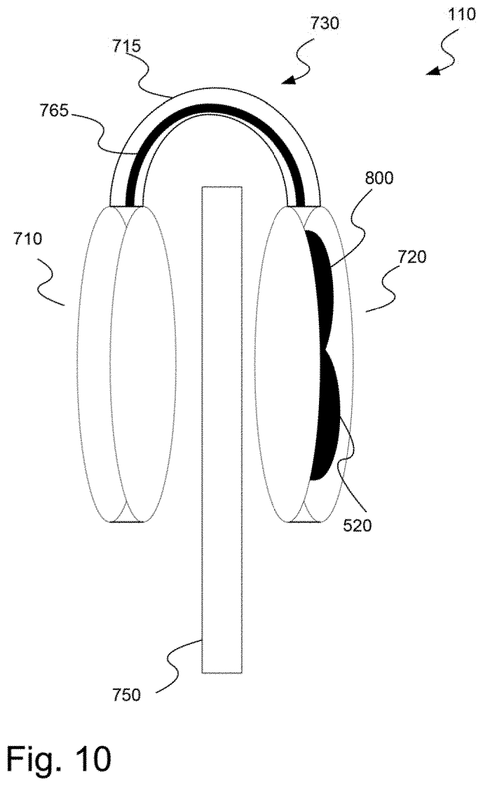

In some embodiments, connector 730 may include a flexible printed circuit board (PCB). FIG. 10 illustrates an exemplary embodiment wherein connector 730 includes a flexible printed circuit board 765. Flexible printed circuit board 765 may include data connections and power connections between capturing unit 710 and power unit 720. Thus, in some embodiments, flexible printed circuit board 765 may serve to replace power cable 760 and data cable 770. In alternative embodiments, flexible printed circuit board 765 may be included in addition to at least one of power cable 760 and data cable 770. In various embodiments discussed herein, flexible printed circuit board 765 may be substituted for, or included in addition to, power cable 760 and data cable 770.

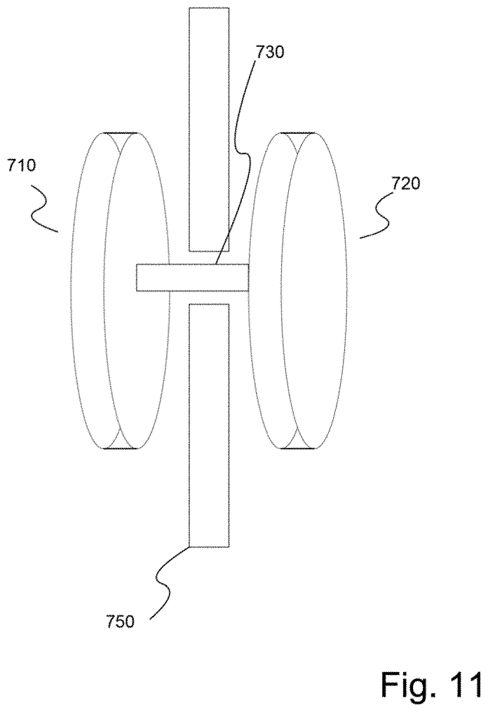

FIG. 11 is a schematic illustration of another embodiment of a wearable apparatus securable to an article of clothing consistent with the present disclosure. As illustrated in FIG. 11, connector 730 may be centrally located with respect to capturing unit 710 and power unit 720. Central location of connector 730 may facilitate affixing apparatus 110 to clothing 750 through a hole in clothing 750 such as, for example, a button-hole in an existing article of clothing 750 or a specialty hole in an article of clothing 750 designed to accommodate wearable apparatus 110.

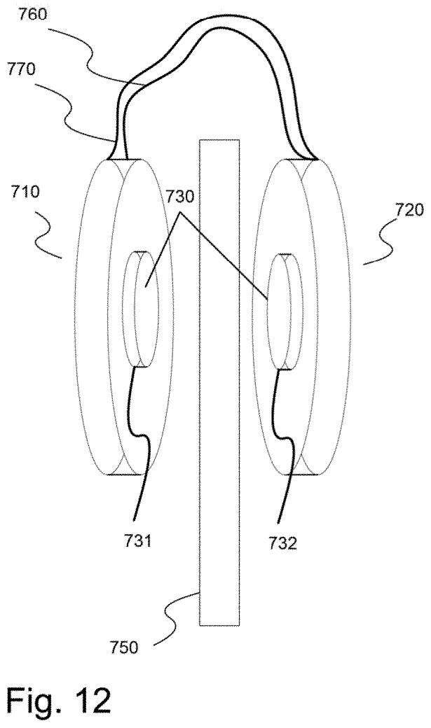

FIG. 12 is a schematic illustration of still another embodiment of wearable apparatus 110 securable to an article of clothing. As illustrated in FIG. 12, connector 730 may include a first magnet 731 and a second magnet 732. First magnet 731 and second magnet 732 may secure capturing unit 710 to power unit 720 with the article of clothing positioned between first magnet 731 and second magnet 732. In embodiments including first magnet 731 and second magnet 732, power cable 760 and data cable 770 may also be included. In these embodiments, power cable 760 and data cable 770 may be of any length, and may provide a flexible power and data connection between capturing unit 710 and power unit 720. Embodiments including first magnet 731 and second magnet 732 may further include a flexible PCB 765 connection in addition to or instead of power cable 760 and/or data cable 770. In some embodiments, first magnet 731 or second magnet 732 may be replaced by an object comprising a metal material.

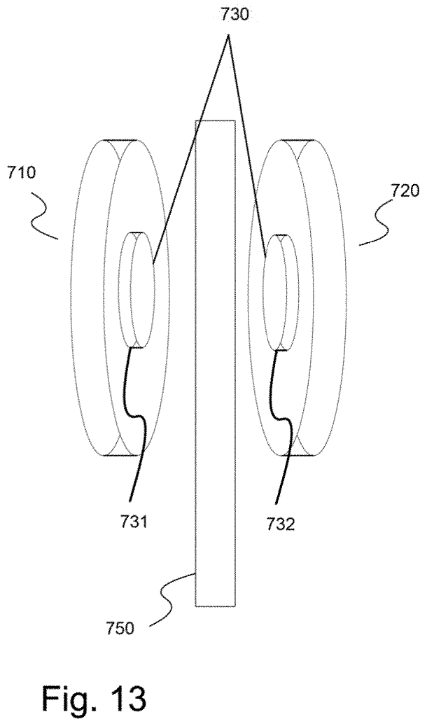

FIG. 13 is a schematic illustration of yet another embodiment of a wearable apparatus 110 securable to an article of clothing. FIG. 13 illustrates an embodiment wherein power and data may be wirelessly transferred between capturing unit 710 and power unit 720. As illustrated in FIG. 13, first magnet 731 and second magnet 732 may be provided as connector 730 to secure capturing unit 710 and power unit 720 to an article of clothing 750. Power and/or data may be transferred between capturing unit 710 and power unit 720 via any suitable wireless technology, for example, magnetic and/or capacitive coupling, near field communication technologies, radiofrequency transfer, and any other wireless technology suitable for transferring data and/or power across short distances.

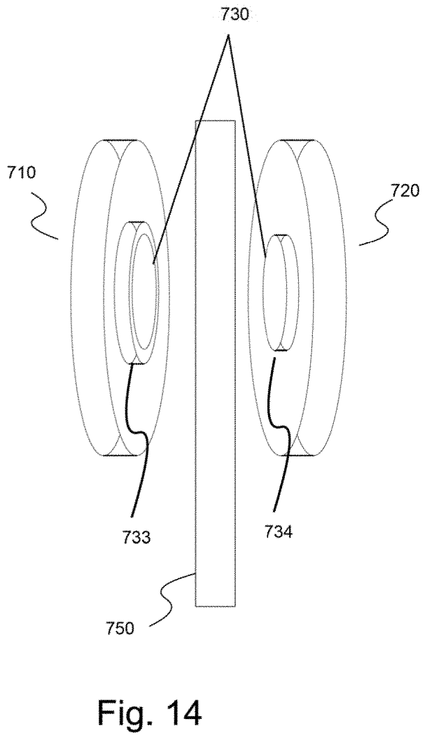

FIG. 14 illustrates still another embodiment of wearable apparatus 110 securable to an article of clothing 750 of a user. As illustrated in FIG. 14, connector 730 may include features designed for a contact fit. For example, capturing unit 710 may include a ring 733 with a hollow center having a diameter slightly larger than a disk-shaped protrusion 734 located on power unit 720. When pressed together with fabric of an article of clothing 750 between them, disk-shaped protrusion 734 may fit tightly inside ring 733, securing capturing unit 710 to power unit 720. FIG. 14 illustrates an embodiment that does not include any cabling or other physical connection between capturing unit 710 and power unit 720. In this embodiment, capturing unit 710 and power unit 720 may transfer power and data wirelessly. In alternative embodiments, capturing unit 710 and power unit 720 may transfer power and data via at least one of cable 760, data cable 770, and flexible printed circuit board 765.

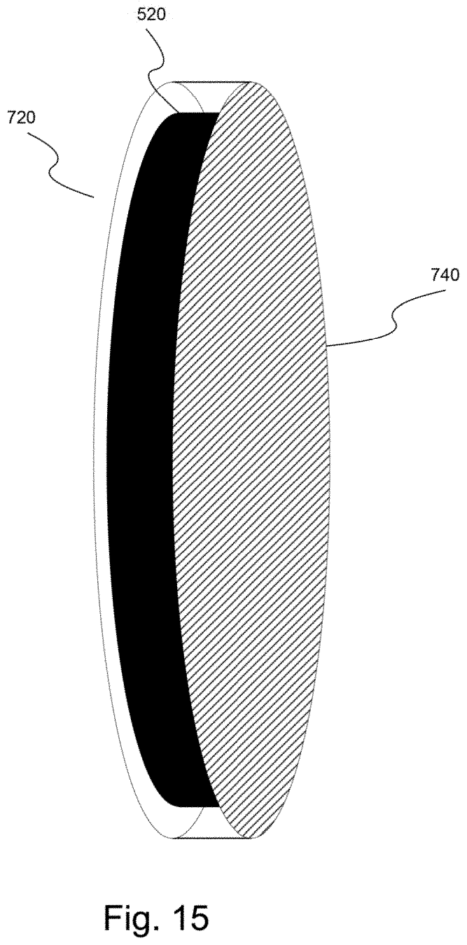

FIG. 15 illustrates another aspect of power unit 720 consistent with embodiments described herein. Power unit 720 may be configured to be positioned directly against the user's skin. To facilitate such positioning, power unit 720 may further include at least one surface coated with a biocompatible material 740. Biocompatible materials 740 may include materials that will not negatively react with the skin of the user when worn against the skin for extended periods of time. Such materials may include, for example, silicone, PTFE, kapton, polyimide, titanium, nitinol, platinum, and others. Also as illustrated in FIG. 15, power unit 720 may be sized such that an inner volume of the power unit is substantially filled by mobile power source 520. That is, in some embodiments, the inner volume of power unit 720 may be such that the volume does not accommodate any additional components except for mobile power source 520. In some embodiments, mobile power source 520 may take advantage of its close proximity to the skin of user's skin. For example, mobile power source 520 may use the Peltier effect to produce power and/or charge the power source.

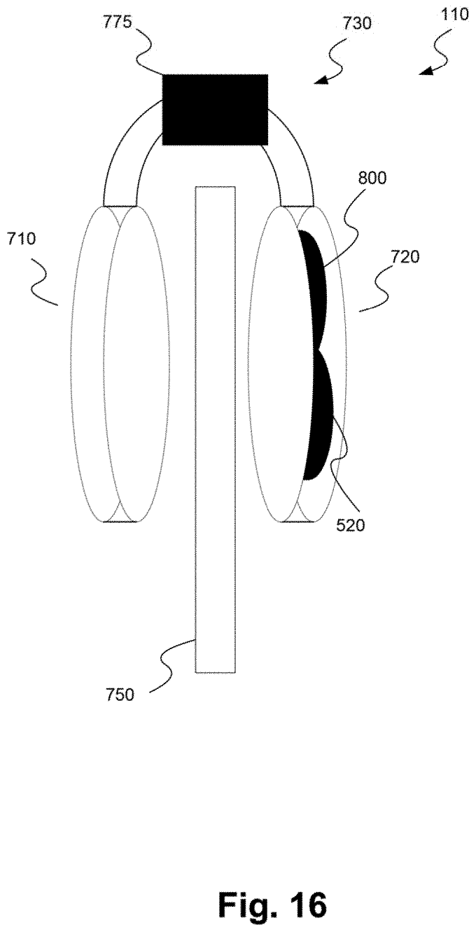

In further embodiments, an apparatus securable to an article of clothing may further include protective circuitry associated with power source 520 housed in in power unit 720. FIG. 16 illustrates an exemplary embodiment including protective circuitry 775. As illustrated in FIG. 16, protective circuitry 775 may be located remotely with respect to power unit 720. In alternative embodiments, protective circuitry 775 may also be located in capturing unit 710, on flexible printed circuit board 765, or in power unit 720.

Protective circuitry 775 may be configured to protect image sensor 220 and/or other elements of capturing unit 710 from potentially dangerous currents and/or voltages produced by mobile power source 520. Protective circuitry 775 may include passive components such as capacitors, resistors, diodes, inductors, etc., to provide protection to elements of capturing unit 710. In some embodiments, protective circuitry 775 may also include active components, such as transistors, to provide protection to elements of capturing unit 710. For example, in some embodiments, protective circuitry 775 may comprise one or more resistors serving as fuses. Each fuse may comprise a wire or strip that melts (thereby braking a connection between circuitry of image capturing unit 710 and circuitry of power unit 720) when current flowing through the fuse exceeds a predetermined limit (e.g., 500 milliamps, 900 milliamps, 1 amp, 1.1 amps, 2 amp, 2.1 amps, 3 amps, etc.) Any or all of the previously described embodiments may incorporate protective circuitry 775.

In some embodiments, the wearable apparatus may transmit data to a computing device (e.g., a smartphone, tablet, watch, computer, etc.) over one or more networks via any known wireless standard (e.g., cellular, Wi-Fi, Bluetooth®, etc.), or via near-filed capacitive coupling, other short range wireless techniques, or via a wired connection. Similarly, the wearable apparatus may receive data from the computing device over one or more networks via any known wireless standard (e.g., cellular, Wi-Fi, Bluetooth®, etc.), or via near-filed capacitive coupling, other short range wireless techniques, or via a wired connection. The data transmitted to the wearable apparatus and/or received by the wireless apparatus may include images, portions of images, identifiers related to information appearing in analyzed images or associated with analyzed audio, or any other data representing image and/or audio data. For example, an image may be analyzed and an identifier related to an activity occurring in the image may be transmitted to the computing device (e.g., the “paired device”). In the embodiments described herein, the wearable apparatus may process images and/or audio locally (on board the wearable apparatus) and/or remotely (via a computing device). Further, in the embodiments described herein, the wearable apparatus may transmit data related to the analysis of images and/or audio to a computing device for further analysis, display, and/or transmission to another device (e.g., a paired device). Further, a paired device may execute one or more applications (apps) to process, display, and/or analyze data (e.g., identifiers, text, images, audio, etc.) received from the wearable apparatus.

Some of the disclosed embodiments may involve systems, devices, methods, and software products for determining at least one keyword. For example, at least one keyword may be determined based on data collected by apparatus 110. At least one search query may be determined based on the at least one keyword. The at least one search query may be transmitted to a search engine.

In some embodiments, at least one keyword may be determined based on at least one or more images captured by image sensor 220. In some cases, the at least one keyword may be selected from a keywords pool stored in memory. In some cases, optical character recognition (OCR) may be performed on at least one image captured by image sensor 220, and the at least one keyword may be determined based on the OCR result. In some cases, at least one image captured by image sensor 220 may be analyzed to recognize: a person, an object, a location, a scene, and so forth. Further, the at least one keyword may be determined based on the recognized person, object, location, scene, etc. For example, the at least one keyword may comprise: a person's name, an object's name, a place's name, a date, a sport team's name, a movie's name, a book's name, and so forth.

In some embodiments, at least one keyword may be determined based on the user's behavior. The user's behavior may be determined based on an analysis of the one or more images captured by image sensor 220. In some embodiments, at least one keyword may be determined based on activities of a user and/or other person. The one or more images captured by image sensor 220 may be analyzed to identify the activities of the user and/or the other person who appears in one or more images captured by image sensor 220. In some embodiments, at least one keyword may be determined based on at least one or more audio segments captured by apparatus 110. In some embodiments, at least one keyword may be determined based on at least GPS information associated with the user. In some embodiments, at least one keyword may be determined based on at least the current time and/or date.

In some embodiments, at least one search query may be determined based on at least one keyword. In some cases, the at least one search query may comprise the at least one keyword. In some cases, the at least one search query may comprise the at least one keyword and additional keywords provided by the user. In some cases, the at least one search query may comprise the at least one keyword and one or more images, such as images captured by image sensor 220. In some cases, the at least one search query may comprise the at least one keyword and one or more audio segments, such as audio segments captured by apparatus 110.

In some embodiments, the at least one search query may be transmitted to a search engine. In some embodiments, search results provided by the search engine in response to the at least one search query may be provided to the user. In some embodiments, the at least one search query may be used to access a database.

For example, in one embodiment, the keywords may include a name of a type of food, such as quinoa, or a brand name of a food product; and the search will output information related to desirable quantities of consumption, facts about the nutritional profile, and so forth. In another example, in one embodiment, the keywords may include a name of a restaurant, and the search will output information related to the restaurant, such as a menu, opening hours, reviews, and so forth. The name of the restaurant may be obtained using OCR on an image of signage, using GPS information, and so forth. In another example, in one embodiment, the keywords may include a name of a person, and the search will provide information from a social network profile of the person. The name of the person may be obtained using OCR on an image of a name tag attached to the person's shirt, using face recognition algorithms, and so forth. In another example, in one embodiment, the keywords may include a name of a book, and the search will output information related to the book, such as reviews, sales statistics, information regarding the author of the book, and so forth. In another example, in one embodiment, the keywords may include a name of a movie, and the search will output information related to the movie, such as reviews, box office statistics, information regarding the cast of the movie, show times, and so forth. In another example, in one embodiment, the keywords may include a name of a sport team, and the search will output information related to the sport team, such as statistics, latest results, future schedule, information regarding the players of the sport team, and so forth. For example, the name of the sports team may be obtained using audio recognition algorithms.

Using a Wearable Apparatus in Social Events

A wearable apparatus consistent with the disclosed embodiments may be used in social events to identify individuals in the environment of a user of the wearable apparatus and provide contextual information associated with the individual. For example, the wearable apparatus may determine whether an individual is known to the user, or whether the user has previously interacted with the individual. The wearable apparatus may provide an indication to the user about the identified person, such as a name of the individual or other identifying information. The device may also extract any information relevant to the individual, for example, words extracted from a previous encounter between the user and the individual, topics discussed during the encounter, or the like. The device may also extract and display information from external source, such as the internet. Further, regardless of whether the individual is known to the user or not, the wearable apparatus may pull available information about the individual, such as from a web page, a social network, etc. and provide the information to the user.

This content information may be beneficial for the user when interacting with the individual. For example, the content information may remind the user who the individual is. For example, the content information may include a name of the individual, or topics discussed with the individual, which may remind the user of how he or she knows the individual. Further, the content information may provide talking points for the user when conversing with the individual, for example, the user may recall previous topics discussed with the individual, which the user may want to bring up again. In some embodiments, for example where the content information is derived from a social media or blog post, the user may bring up topics that the user and the individual have not discussed yet, such as an opinion or point of view of the individual, events in the individual's life, or other similar information. Thus, the disclosed embodiments may provide, among other advantages, improved efficiency, convenience, and functionality over prior art devices.

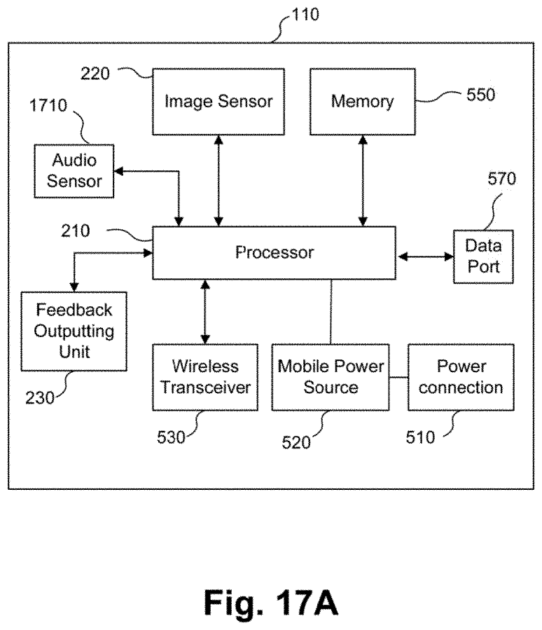

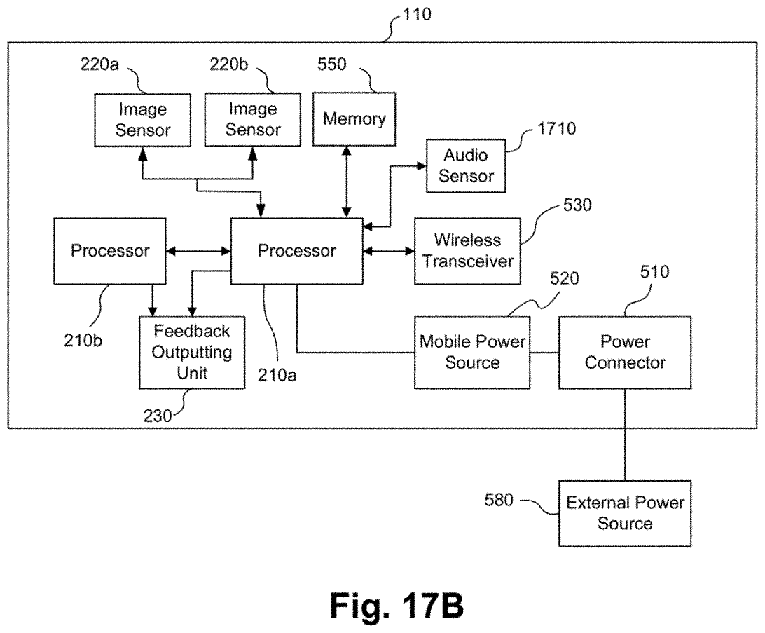

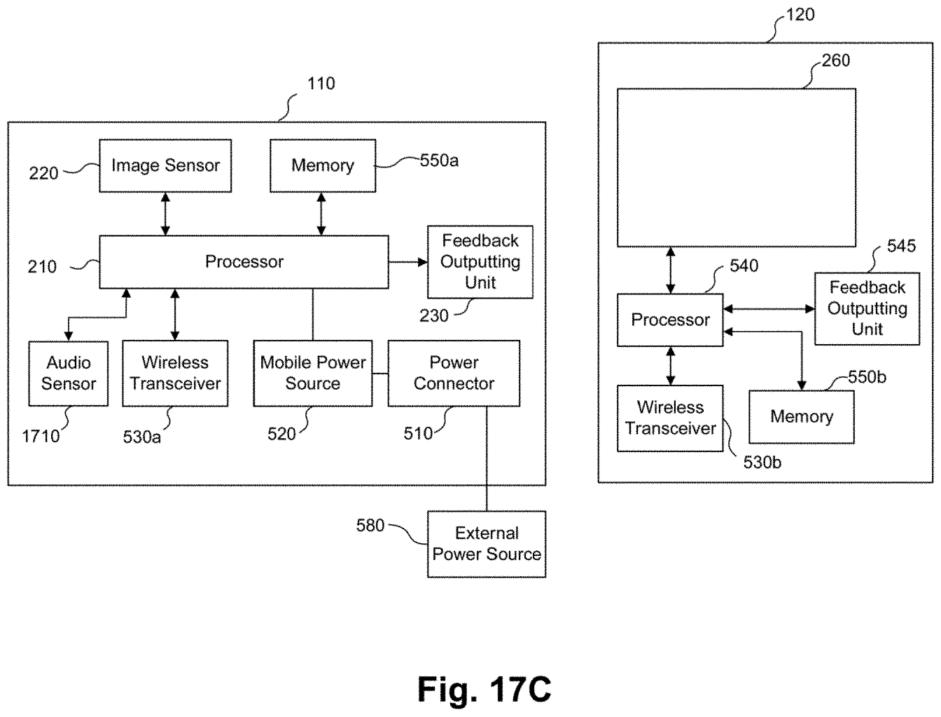

In some embodiments, apparatus 110 may be configured to use audio information in addition to image information. For example, apparatus 110 may detect and capture sounds in the environment of the user, via one or more microphones. Apparatus 110 may use this audio information instead of, or in combination with, image information to determine situations, identify persons, perform activities, or the like. FIG. 17A is a block diagram illustrating components of wearable apparatus 110 according to an example embodiment. FIG. 17A may include the features shown in FIG. 5A. For example, as discussed in greater detail above, wearable apparatus may include processor 210, image sensor 220, memory 550, wireless transceiver 530 and various other components as shown in FIG. 17A. Wearable apparatus may further comprise an audio sensor 1710. Audio sensor 1710 may be any device capable of capturing sounds from an environment of a user and converting them to one or more audio signals. For example, audio sensor 1710 may comprise a microphone or another sensor (e.g., a pressure sensor, which may encode pressure differences comprising sound) configured to encode sound waves as a digital signal. As shown in FIG. 17A, processor 210 may analyze signals from audio sensor 1710 in addition to signals from image sensor 220.

FIG. 17B is a block diagram illustrating the components of apparatus 110 according to another example embodiment. Similar to FIG. 17A, FIG. 17B includes all the features of FIG. 5B along with audio sensor 1710. Processor 210a may analyze signals from audio sensor 1710 in addition to signals from image sensors 210a and 210b. In addition, although FIGS. 17A and 17B each depict a single audio sensor, a plurality of audio sensors may be used, whether with a single image sensor as in FIG. 17A or with a plurality of image sensors as in FIG. 17B.

FIG. 17C is a block diagram illustrating components of wearable apparatus 110 according to an example embodiment. FIG. 17C includes all the features of FIG. 5C along with audio sensor 1710. As shown in FIG. 17C, wearable apparatus 110 may communicate with a computing device 120. In such embodiments, wearable apparatus 110 may send data from audio sensor 1710 to computing device 120 for analysis in addition to or in lieu of analyze the signals using processor 210.

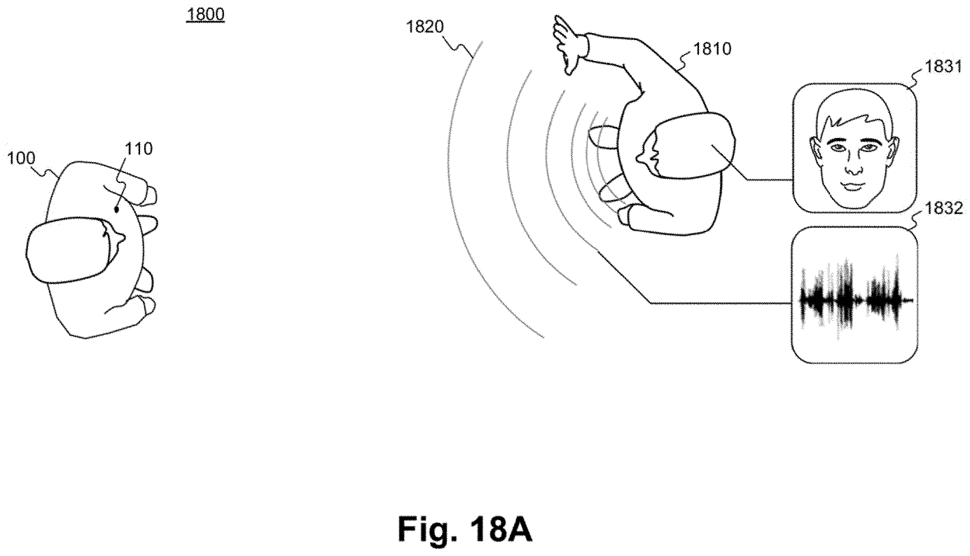

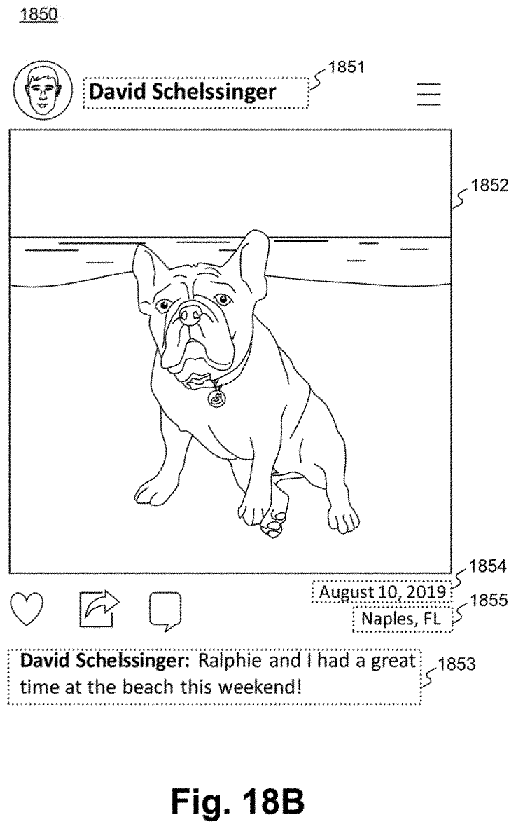

FIG. 18A illustrates an example environment 1800 in which a user 100 may interact with an individual 1810, consistent with the disclosed embodiments. User 100 may be wearing apparatus 110, as shown, which may correspond to the wearable apparatus 110 shown in FIGS. 17A-17C. As discussed above, apparatus 110 may be worn by user 100 in various configurations, including being physically connected to a shirt, necklace, a belt, glasses, a wrist strap, a button, or other articles associated with user 100. In some embodiments, one or more additional devices may also be included, such as computing device 120. Accordingly, one or more of the processes or functions described herein with respect to apparatus 110 or processor 210 may be performed by computing device 120 and/or processor 540. As shown in FIG. 18A, user 100 may be in the same environment as another individual 1820. In some embodiments, user 100 may be engaging in (or about to engage in) a conversation with individual 1810. In other embodiments, user 100 and individual 1810 may not be engaged in a conversation but individual 1810 may be within view of user 100 (or wearable apparatus 110).

Apparatus 110 may capture images or other information from environment 1800. For example, image sensor 220 may capture images including a representation of individual 1810. In some embodiments, apparatus 110 may further capture sound from environment 1800. For example, individual 1810 may be speaking and may generate sound 1820. Audio sensor 1710, which may comprise a microphone, may capture sound 1820 and may convert it to an audio signal to be processed by processor 210.

Based on the captured images and/or audio, wearable apparatus 110 may be configured to determine contextual information associated with individual 1810 and provide the contextual information to user 100. In some embodiments, this may include determining whether individual 1810 is a recognized individual of user 100. For example, this may include determining whether individual 1810 is included in or otherwise associated with a contact list of user 100, determining whether user 100 has previously seen or engaged with individual 1810, determining whether individual 1810 is included in or associated with a social network of user 100, etc.

Processor 210 may be configured to recognize identifying features of individual 1810 from the images and the audio signals. For example, processor 210 may use one or more image recognition techniques to extract visual features 1831 from one or more images that are associated with individual 1810. Visual features 1831 may include facial features of individual 1810, as depicted in FIG. 18A, such as the eyes, nose, cheekbones, jaw, or other features. It is understood that features 1831 are not limited to facial features, however and may include any physical features of individual 1810 which may be used for identification (e.g., size, body shape, posture, clothing, nametags, etc.) The extracted features may be analyzed to determine an identity of individual 1810. Processor 210 may use one or more algorithms for analyzing the detected features, such as principal component analysis (e.g., using eigenfaces), linear discriminant analysis, elastic bunch graph matching (e.g., using Fisherface), Local Binary Patterns Histograms (LBPH), Scale Invariant Feature Transform (SIFT), Speed Up Robust Features (SURF), or the like.

In some embodiments, processor 210 may be configured to analyze the audio signals received from audio sensor 1710 to identify individual 1810. Processor 210 may be configured to use one or more voice recognition algorithms (e.g., Hidden Markov Models, Dynamic Time Warping, neural networks, or other techniques) to recognize the individual by his or her voice. Processor 210 may identify various vocal characteristics 1832 associated with individual 1810, such as an accent, a speech pattern, an approximate age, a gender, or the like.

Processor 210 may use the images and/or audio signals to determine whether individual 1810 is known to user 100. In some embodiments, processor 210 may compare the captured images and/or audio signals (or visual features 1831 and/or vocal characteristics 1832) to a database. The database may be stored locally on apparatus 110 (e.g., in memory 550), in a device associated with apparatus 110, such as computing device 120 (e.g., in memory 550b), or in a remote storage location (e.g., accessed through wireless transceiver 530). The database may include a list of individuals known to user 100. For example, a contact list may be associated with a mobile device (e.g., computing device 120) of user 100 and may contain images associated with the contacts which may be used to identify individual 1810. In some embodiments, the database may be associated with a social network platform, such as Facebook™, LinkedIn™, Instagram™, etc. and processor 210 may compare the image and/or audio data with data (e.g., friends lists, connections, etc.) stored in the social network platform to determine whether individual 1810 is known to user 100.

In some embodiments, the database may be a historical list of individuals that user 100 has encountered and/or interacted with. For example, each time user 100 meets an individual, is introduced to an individual, observes an individual (e.g., attends a meeting with the individual, observes a conversation between the individual and others, etc.), or otherwise interacts with the individual, apparatus 110 may be configured to store information associated with the individual in a database. In some embodiments, apparatus 110 may store a name of the individual, which may be obtained from audio signals (e.g., if the name of the individual is spoken), by text recognition (e.g., from a nametag in an image, etc.), through manual entry (e.g., by user 100 through computing device 100), or the like. Apparatus 110 may store other information, such as visual features 1831 or vocal characteristics 1832, which may be used to identify the individual in future encounters. Apparatus 110 may further store information pertaining to the encounter. For example, apparatus 110 may transcribe spoken words associated with the individual (e.g., a conversation between the individual and user 100 or between the individual and others, a speech by the individual, etc.) and may store the transcribed words or recorded audio for future reference. In some embodiments apparatus 110 may determine and store one or more topics of conversation based on the transcribed conversation. For example, processor 210 may identify various keywords such as “golf,” “fairway,” “handicap,” “teebox,” “driver,” etc. and may store “golf” as a topic of conversation. Processor 210 may build on this database by storing information associated with later encounters with the same individual and attributing them to the same individual within the database. Based on the stored information, processor 210 may determine whether individual 1810 is known to user 100. For example, processor 210 may compare visual features 1831 and/or vocal characteristics 1832 to information stored in the database to determine whether individual 1810 is known to user 100.

In some embodiments, processor 210 may be configured to determine a level of confidence associated with the identification of individual 1810. The level of confidence may be based on the degree of match between the identified visual features 1831 and/or vocal characteristics 1832 and the information stored in the database. The level of confidence may be represented as a percentage (e.g., 0%-100% match), on a predefined scale (e.g., 1-10), through predefined confidence levels (e.g., low confidence, high confidence, etc.), or the like. For example, if the detected facial features associated with individual 1810 match closely but not completely with stored facial features of a known individual, individual 1810 may be identified as the known individual with a confidence score of 90%. The confidence score may also be based on the amount or quality of information available to processor 210. For example, if individual 1810 is far away and therefore a relatively low resolution image is used, a lower confidence score may be assigned. Similarly, if only a short audio signal is captured, this may also result in a lower confidence score. In some embodiments, processor 210 may identify multiple possible recognized individuals and give an associated confidence score for each.

Processor 210 may further be configured to determine a content item associated with the individual. The content item may include any accessible information that may be relevant to the user when encountering individual 1810. In instances where processor 210 determines that individual 1810 is a recognized individual, the content item may be accessed from a contact list, a social network platform, a database, etc. When individual 1810 is not identified as a recognized individual, the content item may be retrieved from an external content source associated with the individual. For example, the content item may be accessed from a webpage, a blog, a social media network, or the like. Processor 210 may perform an image search based on a representation of individual 1810 from the captured images (which may include visual features 1831). Processor 210 may perform a name search based on a name of individual 1810 as identified vocally or visually. The image or name search may return results associated with individual 1810, such as a blog, a vlog (video blog), a social media page, a personal or company website, etc., from which the content item may be extracted. In some embodiments, multiple searches may be performed. For example, processor 210 may first perform an image search to identify a name of individual 1810 and may then search using the name of individual 1810 to access the content source. In some embodiments, the search may be performed by one or more processing units other than processor 210 (e.g., processor 540 of computing device 120) and processor 210 may provide instructions for performing the search.

In some embodiments, the content item may include a name or other identifying information of individual 1810, such as a title, a company or organization associated with the individual. For example, the content item may identify individual 1810 as “Dave Schlessinger, Lead Product Engineer at TwistLace, Inc.” In some embodiments, the content item may further include contextual information relative to the environment of the user. For example, the content item may indicate that “Dave Schlessinger is in the room” or “Dave Schlessinger is in front of you at approximately 10 meters,” etc. The content item may include various other information, such as a relationship to the user, a relationship to other individuals known to the user, biographical information (e.g., a birthdate, etc.), a stored image of individual 1810, a vocal pronunciation of the name of individual 1810, a name of a spouse of individual 1810, names of children of individual 1810, a nickname of individual 1810, or any other information that may be relevant to user 100.

In instances where individual 1810 is determined to be an individual known to user 100, the content item may include information associated with a previous encounter with individual 1810. For example, processor 210 may be configured to access a database storing information pertaining to previous encounters between the user and a plurality of individuals. In some embodiments, the content item may comprise information associated with a previous conversation between the user and individual 1810. For example, the content item may include one or more topics of conversation in the previous encounter. As discussed above, processor 210 may be configured to automatically identify topics of conversation based on a transcript of the conversation, which may be generated by processor 210 based on audio recorded by audio sensor 1710 or received from another source. The topic of conversation may be determined by identifying keywords within the transcribed conversation and associating the keywords with a topic. In some embodiments, the topic may be identified through a trained machine learning algorithm. For example, the algorithm may be trained using a training set of recorded or transcribed conversations associated with known topics to develop a model which may be used to identify topics in other conversations. As one example, individual 1810 may tell user 100 that his daughter just started playing ice hockey this season. In this example, processor 210 may extract and store topics such as “daughter” and/or “ice hockey” which may be returned as the content item in a later encounter with individual 1810. In some embodiments, the content item may include a topic sentence, such as “Dave's daughter plays ice hockey,” which may be generated based on the transcript of the conversation and/or the determined topics. When presented with these topics, user 100 may be reminded who individual 1810 is, or may be prompted to ask individual 1810 about how his daughter is enjoying hockey.

In some embodiments, the content item may include information from multiple previous conversations (e.g., the name of individual 1810's daughter, other sports individual 1810 is interested in, activities of other children of individual 1810, etc.). In some embodiments, the topic of conversation or notes pertaining to the conversation may be manually entered by a user. For example, after a conversation with individual 1810, user 100 may enter notes such as “discussed Dave's new position at TwistLace, Inc.,” or similar notes pertaining to individual 1810 or the conversation. In some embodiments, the notes and/or topics may be automatically generated and presented to user 100 (e.g., through computing device 120). User 100 may then select which topics or notes should be recorded and may edit the topics or notes before they are stored. These notes and/or topics of conversation may be retrieved as the content item.

Various other information associated with the previous encounter may be included in the content item. For example, the content item may comprise a date and/or time of the last encounter between user 100 and individual 1810. In some embodiments, the content item may include a location of the last encounter, which may be determined based on GPS data obtained during the encounter (e.g., by apparatus 110, computing device 120, or an external device such as a smartphone, a smart watch, a fitness tracker, etc.). The content item may include names of other individuals present during the encounter, a context of the encounter (e.g., March 2019 product development meeting, dinner at Dave's house, etc.), physical properties of individual 1810 (e.g., height, hair color, hairstyle, etc.), or any other relevant information. In some embodiments, the content item may include all or a portion of the previous conversation with individual 1810. For example, the content item may be an audio clip or a snippet of a transcript of a conversation with individual 1810.

In some embodiments, the previous encounter may be an electronic communication between user 100 and individual 1810. Processor 210 may be configured to access stored conversations between user 100 and individual 1810 and extract content items from the stored conversations. For example, the electronic communications may be in the form of an email exchange, a text message (e.g., an SMS or MMS message), a messaging platform (e.g., Facebook Messenger™, Whatsapp™, Telegram™, etc.). As with the in-person conversations discussed above, the content item may include a topic of conversation in the electronic communication, a snippet of the conversation, or the like. In some embodiments, the content item may also include a file attached to or included in the communication. For example, the content item may include an image or other document sent between user 100 and individual 1810. In some embodiments the communications may be accessed from a remote resource, such as a server, or from an internal device memory, including memory 550 or 550a of apparatus 110, memory 550b of computing device 120, a memory of another associated device, or the like.

In some embodiments, the content item may be retrieved from an external content source, as discussed above. This may be true regardless of whether individual 1810 is known to user 100. For example, if individual 1810 is known to user 100, processor 210 may access an external source that has been linked or associated with individual 1810. Where individual 1810 is not known to user 100, the external source may be accessed through a search, for example, based on visual features 1831 and/or vocal characteristics 1832 of individual 1810, as discussed above. The external source may include any accessible source of information that is remote from apparatus 110 and/or computing device 120. In some embodiments, the external source may be an internet source such as a webpage. For example, the webpage may be a blog hosted by individual 1810, a blog associated with individual 1810 (e.g., a blog in which individual 1810 is an active member, posts to a discussion board, etc.), a company website, a personal website, or the like. The content source may also be a social media platform in which individual 1810 has an account or interacts with. For example, the content source may include an account or profile associated with Facebook™, Twitter™, LinkedIn™, YouTube™, Instagram™, Tumblr™, Reddit™, or other social media platforms. In some embodiments, the content item may include profile information associated with the external source. For example, the content item may include a name of individual 1810, a username, a birthdate, a “bio” or biographical summary, a location, or the like which may be extracted from the webpage or social media profile.

In some embodiments, the content item may include posts by individual 1810 or posts by others on the external source that are associated with individual 1810 (e.g., where individual 1810 has “liked” the post, is mentioned in the post, where individual 1810 has commented on the post, etc.). FIG. 18B illustrates an example social media post 1850 that may be used to determine a content item consistent with the disclosed embodiments. As discussed above, social media post 1850 may be located on a blog, a vlog, a webpage, a social media platform or the like. In some embodiments the entire social media post may be presented as the content item (e.g., a link to the post, an image of the post, etc.). In other embodiments, processor 210 may extract information from social media post 1850 and present it to user 100 as the content item. For example, the content item may include a name 1851 extracted from social media post 1850 or from an associated account. This may be helpful, for example, if user 100 does not know the name of individual 1810 or does not remember it. In some embodiments, processor 210 may be configured to analyze an image 1852 associated with social media post 1850. For example, image 1852 may be an image taken by and/or posted by individual 1810. Processor 210 may perform image recognition techniques to extract information from image 1852, which may provide additional information to be included in the content item. For example, processor 210 may determine that image 1852 contains a dog (or more specifically, a French bulldog), a beach, etc., and the content item may indicate to user 100 that individual 1810 has a French bulldog or that individual 1810 went to the beach. This may be helpful to user 100 for remembering who individual 1810 is, or for reminding user 100 to ask about individual 1810's dog or recent vacation. In some embodiments, image 1852 itself may be included in the content item.

Processor 210 may be configured to analyze text 1853 associated with social media post 1850 to extract information. Text 1853 may include text written by individual 1810 (as shown in FIG. 18A), or other text (e.g., comments by others in response to the post, etc.). In the example shown in FIG. 18A, text 1853 may be analyzed to determine that individual 1810's dog is named “Ralphie,” which may also be included in the content item. In some embodiments, text 1853 itself may be included in the content item. Processor 210 may analyze other information included in the post, such as date 1854, which may indicate when individual 1810 was at the beach and may be included in the content item. Social media post 1850 may further include a location 1855, which may be included in the content item. Various other properties of social media post 1850 may also be analyzed and/or included in the content item, such as a number of “likes” or other signals the post has received, a number of comments associated with the post, or other properties.

In some embodiments the data extracted from social media post 1850 may be processed further to generate a note. For example, based on the features identified above, processor 210 may generate a note such as “Dave has a French bulldog named Ralphie” or “Dave visited Naples, Fla. in August,” which may be included in the content item. In some embodiments, information may be extracted from multiple social media posts and from multiple webpages or social media platforms. While a personal social media post is used as an example in FIG. 18A, it is understood that the posts may be in a professional or scholarly context. For example, the social media post may be a LinkedIn™ post, a post on a company or professional webpage, a post from a collaborative work platform (e.g., a Sharepoint™ site, etc.) or various other forms of social media posts. Other information that may be extracted and included in the content item may include a political stance of individual 1810, an opinion of individual 1810, a favorite sports team of individual 1810, a university attended by individual 1810, a restaurant or other location individual 1810 has visited, research conducted by individual 1810, or the like. User 100 can then refer to the content item and use it to start a conversation, promote common interests, etc.

The content item may be presented to user 100 in various ways. In some embodiments, the content item may be visually presented to user 100. For example, the content item may be displayed on a device associated with user 100, such as computing device 120, a smartphone, a wearable device (e.g., a smartwatch, etc.), a laptop computer, a desktop computer, a tablet, etc. In some embodiments, the content item may be presented audibly to user 100. For example, the content item may be presented through a speaker of apparatus 110. In other embodiments, the content item may be presented audibly through a speaker of an external device, including the devices described above. In some embodiments, the external device may include a hearing aid device, which may be placed in or near an ear of user 100, and the content item may be transmitted to the hearing aid device and presented to user 100 through the hearing aid device.

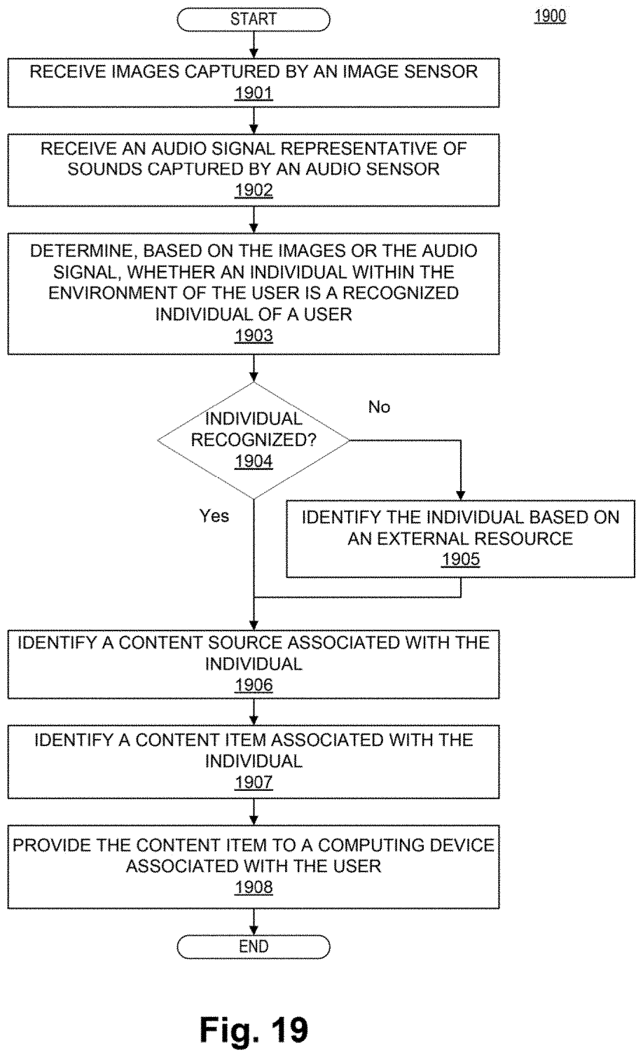

FIG. 19 is a flowchart showing an exemplary process 1900 for using a wearable apparatus in social events consistent with the disclosed embodiments. Process 1900 may be performed by at least one processing device, such as processor 210. Some or all of process 1900 may be performed by processors associated with other components, such as computing device 120 (e.g., processor 540), server 250, and/or other devices.

At step 1901, process 1900 may include receiving a plurality of images captured from an environment of a user of a wearable apparatus by an image sensor. For example, the plurality of images may be received from image sensor 220 and may reflect environment 1800 of user 100. The plurality of images may include a representation of an individual, such as individual 1810, within environment 1800.

At step 1902, process 1900 may include receiving an audio signal representative of a sound captured from the environment of the user by an audio sensor. For example, audio sensor 1710 may capture sound 1820 from environment 1800 and may convert it to an audio signal for processing by processor 210. As discussed above, sound 1820 may represent a voice of individual 1810.

At step 1903, process 1900 may include determining, based on at least one of the plurality of images or the audio signal, whether an individual within the environment of the user is a recognized individual of the user. In some embodiments, step 1903 may include analyzing the plurality of images to extract visual features of the individual, such as visual features 1831, as discussed above. Additionally, or alternatively, step 1903 may include analyzing the audio signal to determine vocal characteristics 1832 of the individual. Determining whether the individual is recognized may comprise comparing the plurality of images (or visual features 1831) and/or the audio signal (or vocal characteristics 1832) to a database to determine the identity of the individual. In some embodiments, step 1903 may include processing the audio signal to extract a spoken name of the individual, which may be used in determining whether the individual is a recognized individual of the user.

As illustrated by step 1904, process 1900 may include different actions depending on whether the individual is recognized. If the individual is not recognized, at step 1905, process 1900 may include, subject to a determination the individual is not a recognized individual, identifying the individual based on an external resource. As described above, identifying the individual based on the external resource may comprise performing an image search based on a representation of the individual depicted in the plurality of images. In some embodiments, this may include performing multiple searches. For example, step 1905 may include performing a first search based on a representation of the individual depicted in the plurality of images to determine a name or other identifying information of the individual, and perform a second search based on the identifying information. At step 1906, process 1900 may include identifying a content source associated with the individual. The content source may be an external content source, for example, one that is accessed through a network. At step 1907, process 1900 may include identifying a first content item associated with the individual. For example, the content source may comprise a social network platform, and the first content item may comprise one or more posts associated with the individual on the social network platform. The post may correspond to social media post 1850, as described above, which may be used to extract information associated with the individual. In other embodiments, the content source may comprise a blog, and the first content item may comprise one or more posts associated with the individual on the blog. Similar to with social media post 1850, processor 210 may be configured to extract information from the blog post to retrieve and/or derive the first content item.

If the individual is recognized at step 1904, process 1900 may include additional actions, such as retrieving, subject to a determination that the individual is a recognized individual, a second content item associated with a previous encounter between the user and the individual and providing the second content item to the computing device associated with the user. For example, the previous encounter may comprise a previous conversation between the user and the individual. Accordingly, the second content item may comprise a topic of conversation associated with the previous conversation, as discussed in greater detail above. The second content item may comprise at least a partial transcript of the previous conversation. For example, the second content item may include an audio clip of the previous conversation or at least a snippet of a transcript of the previous conversation. In some embodiments, the second content item may comprise at least one of a name or a vocal pronunciation of a name of the individual. As discussed above, information regarding previous encounters between the user and the individual may be stored in a database. Accordingly, the second content item may be retrieved from a memory of the wearable apparatus. Alternatively, the second content item may be retrieved from a network storage location, such as a server or cloud storage platform.

At step 1908, process 1900 may include providing the first content item (and/or the second content item) to a computing device associated with the user. In some embodiments, the computing device may be computing device 120, as described above. Accordingly, the computing device may be a mobile phone or other mobile device associated with user 100. The computing device may be configured to display the first content item (and/or the second content item) to the user. In some embodiments, the computing device may be a hearing aid device, which may be configured to audibly present the first content item or the second content item to user 100. User 100 may use the first or second content item to recognize the individual or to inform a discussion between user 100 and the individual. For example, the user may use the content item to strike up a conversation, find common interests, etc.

Using a Wearable Apparatus for Identification

A wearable apparatus consistent with the disclosed embodiments may be used in situations where identification of an individual may be required or desirable as part of a task or routine. As an illustrative example, the wearable apparatus may be used by a delivery person when delivering a parcel to a customer. As part of completing the delivery, authentication of the delivery recipient may be required or preferred. Traditionally, this may be accomplished through asking the recipient of the parcel for his or her name to ensure it matches a name associated with the shipment information. In some instances, the delivery person may also ask for an ID of the recipient to verify the recipient matches information associated with the shipment. In some instances, the delivery person may also require a signature of the recipient which may serve as proof that the delivery was made.

These traditional approaches may increase the time for each delivery to be made while often providing minimal advantages with respect to verification of the recipient. For example, verification of the recipient based on asking for the recipient's name alone can easily be falsified, for example, if an unintended recipient knows the name of the person who lives at the address. Even verification based on a photo ID may be falsified as an unintended recipient may present a fake photo ID where the image matches the unintended recipient and the name matches the person who lives at the delivery address. The delivery person often has no means for comparing the appearance of the actual recipient with an appearance of the intended recipient. Further, signatures may be useful as proof that the parcel was delivered but may not necessarily provide increased authentication. Moreover, even to the extent that these techniques do provide advantages for verifying the recipient, they add to the time for each delivery, which may slow the delivery person on his or her route and may add increased costs associated with the delivery.

Using the disclosed embodiments, a delivery person may be equipped with a wearable apparatus 10. Prior to leaving for a round of deliveries, apparatus 110 may be loaded with the images of each of the clients to be visited in the round. When delivering the parcel to the client, the delivery person may have the option to capture an image of the client receiving the parcel. If the image of the recipient captured through apparatus 110 is verified to be the same person whose image was loaded to apparatus 110, there may be no need for acquiring an identification, signing, or the like. Accordingly, the disclosed methods may provide increased security, functionality, and efficiency over prior art techniques.

While the example of delivering a parcel is used throughout the present disclosure, it is to be understood that this is provided by way of example only. Similar techniques for using a wearable apparatus for identification may be used in a variety of other situations. For example, the disclosed embodiments may be used for verifying the identity of someone picking up an order, for example, from a restaurant or a retail store, or someone picking up a drug prescription from a pharmacy. The disclosed embodiments may be used by medical professionals, such as a doctor or nurse for identifying a patient. In some embodiments, the disclosed methods may be used for admission to a facility, such as verifying the identity of a customer having made a reservation at a restaurant or for verifying the identity of a ticket holder (e.g., for entry into a concert, sporting event, etc.), or the like. The disclosed embodiments may also be used for allowing a passenger to board a transportation vehicle (e.g., an airplane, train, bus, taxi, ridesharing service, etc.), serving notice of a legal action or jury summons, or any other situation where identification may be required or preferred.

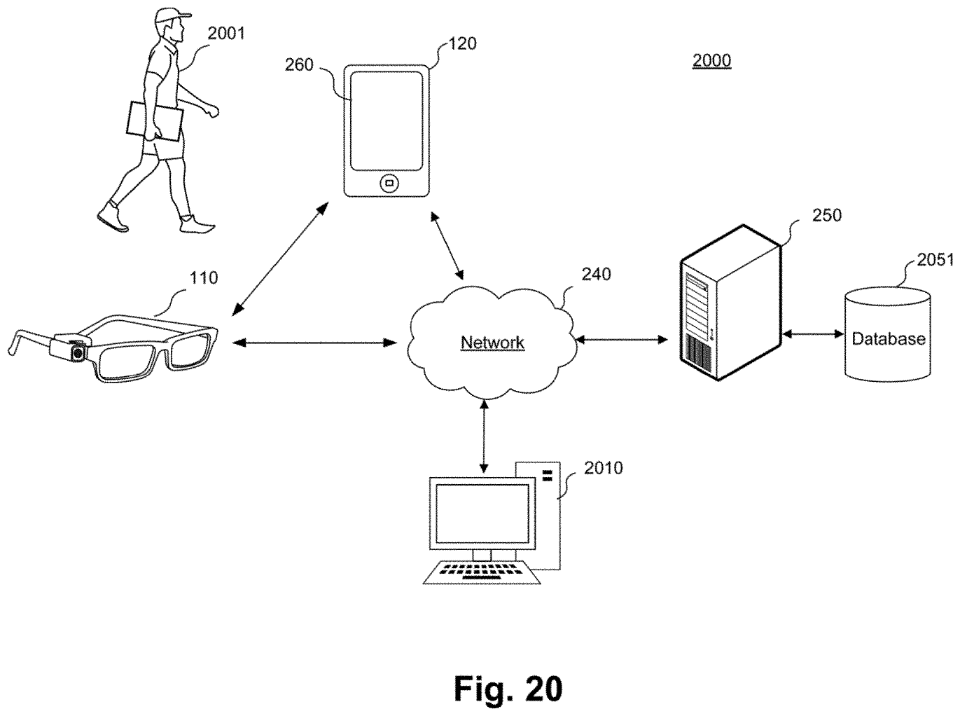

FIG. 20 is a schematic illustration of an example system 2000 used for identification consistent with the disclosed embodiments. System 2000 may include many of the same or similar components to system 200 described above. For example, system 2000 may include a wearable apparatus 110, and an optional computing device 120 and/or a server 250 capable of communicating with apparatus 110 via a network 240. Apparatus 110 may correspond to apparatus 110 as shown in FIGS. 17A-17C, as discussed above. Accordingly, apparatus 110 may include an audio sensor 1710, which may be configured to capture sounds from an environment of user 100 and generate audio signals based on the captured sounds. In the example of a parcel delivery service, apparatus 110 may be worn by a delivery person 2001 (which may correspond to user 100). Delivery person 2001 may be an employee of a parcel delivery service and may use apparatus 110 and/or computing device 120 to aid in or enhance delivery of parcels to customers.

As described above, computing device 120 may include a PC, laptop, tablet, a smartphone, or other computing devices configured to communicate directly with apparatus 110 or server 250 over network 240. Computing device 120 may include a display 260, as shown in FIG. 20. In some embodiments, computing device 120 may be a computing system configured particularly for apparatus 110 and may be provided integral to apparatus 110 or tethered thereto. Apparatus 110 may connect to computing device 120 over network 240 via any known wireless standard (e.g., Wi-Fi, Bluetooth®, etc.), as well as near-field capacitive coupling, and other short-range wireless techniques, or via a wired connection. In the example of a parcel delivery service, delivery person 2001 may carry computing device 120 when making deliveries along a route. In some embodiments computing device 120 may be a mobile phone of delivery person 2001, such as a personal phone, a company phone, etc. In some embodiments, computing device 120 may be a handheld device dedicated to parcel tracking and/or delivery. For example, computing device 120 may be a handheld device designed to be carried by delivery person 2001 for tracking routes and/or deliveries, displaying shipment information, etc. Computing device 120 may include a barcode scanner, a GPS chip, or various other components to facilitate tracking deliveries.

System 2000 may include a client device 2010 configured to communicate with server 250 (or various other components of system 2000) through network 240. Client device 2010 may be any computing device capable of transmitting information to server 250 through a network. Client device 2010 may include devices similar to those described with respect to computing device 120. For example, client device 2010 may include a PC, laptop, tablet, wearable device (e.g., a smartwatch, fitness tracker, etc.), an IOT (Internet-of-Things) device (e.g., a security system, a connected doorbell, tv, etc.), or various other computing devices. In some embodiments, client device 2010 may communicate with server 250 through a network connection separate than that used by apparatus 110 and/or computing device 120 to communicate with server 250. For example, client device 2010 may communicate with server 250 through an internet connection, where apparatus 110 and/or computing device 120 may communicate with server 250 through a secure or dedicated channel. Client device 2010 may be a device used by an intended parcel recipient for placing orders, providing shipping information, tracking shipment information, etc.

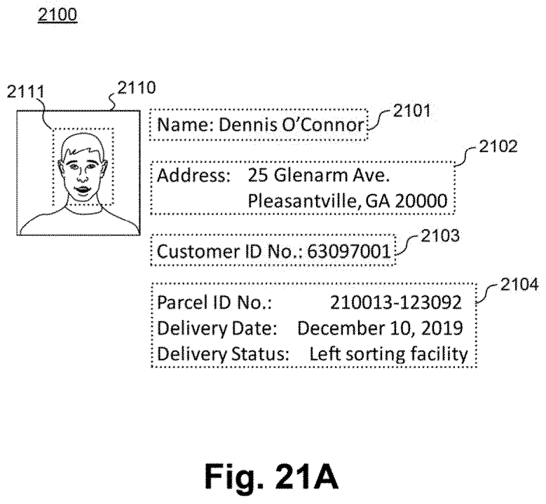

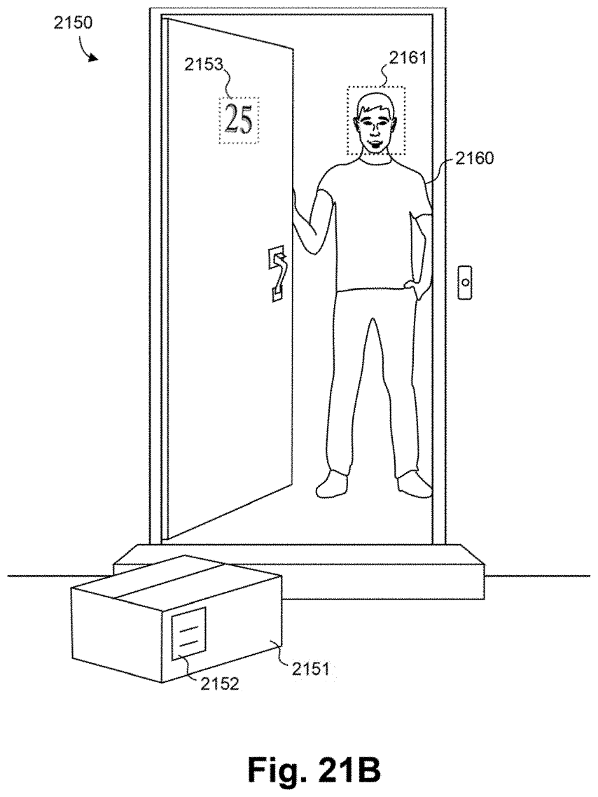

Server 250 may be configured to access a database 2051, which may store information regarding the identity of parcel recipients. In some embodiments, database 2051 may be integral to server 250 or may be accessed by server 250 remotely (e.g., as a separate server, cloud-based storage, etc.). Database 2051 may store a plurality of profiles or entries associated with individuals, which may be customers or intended parcel recipients. For example, database 2051 may associate a name of an intended parcel recipient with data such as image data, a delivery address, parcel information, or the like. FIG. 21A illustrates an example profile 2100 that may be stored in database 2051 consistent with the disclosed embodiments. Profile 2100 may include information such as a name 2010 associated with the individual or an address 2102 associated with the individual. Profile 2100 may include additional identifying information, such as a customer ID number 2103, which may be used for tracking parcels and/or orders associated with the customer. Database 2051 may store historical deliveries or orders associated with the individual as well as current deliveries or orders in progress. In some embodiments, profile 2100 may include parcel information 2014, which may include tracking information for parcels to be delivered to the recipient.

Profile 2100 may include at least one image 2110 of the individual. In some embodiments, image 2110 may be submitted by the individual. For example, the individual may capture an image using client device 2010 (e.g., using a smartphone, tablet, laptop, etc.) and may upload it to server 250. In some embodiments, the individual may upload image 2110 to server 250 from a storage device, which may be included in client device 2010 or may be a separate device. In some embodiments, image 2110 and other information included in profile 2100 may be received by server 250 over a network (e.g., network 240). For example, the individual may create a profile or otherwise provide the information when placing an order with an online retailer or merchant. The online retailer may then transmit the information to the parcel delivery service along with the order information. In some embodiments, the delivery service may combine information from multiple retailers or merchants. For example, if the delivery service receives order information associated with an individual from a first retailer and later receives order information associated with the individual from a second retailer, the delivery service may include the information from both retailers in the same profile 2100 for the individual.

In some embodiments, database 2051 may further store characteristics of the image, such as visual features 2111. For example, server 250 may use one or more image recognition techniques to extract visual features 2111 from the image that are associated with the individual. Visual features 2111 may include facial features of the individual such as the eyes, nose, cheekbones, jaw, or other features. It is understood that visual features 2111 are not limited to facial features and may include any physical features of individual 1810 which may be used for identification (e.g., size, body shape, posture, clothing, nametags, etc.) The extracted features may be associated with the individual in profile 2100. Server 250 may use one or more algorithms for analyzing the detected features, such as principal component analysis (e.g., using eigenfaces), linear discriminant analysis, elastic bunch graph matching (e.g., using Fisherface), Local Binary Patterns Histograms (LBPH), Scale Invariant Feature Transform (SIFT), Speed Up Robust Features (SURF), or the like. In some embodiments, multiple images 2110 or visual features 2111 may be stored.

The information stored in profile 2100 may be used for identifying parcel recipients during delivery. For example, delivery person 2001 may wear apparatus 110 during a delivery route. Apparatus 110 may receive images associated with intended recipients along the route, such as image 2110, and/or characteristics of the images, such as visual features 2111. Apparatus 110 may receive other information, including name 2101, address 2102, customer ID number 2013, and/or parcel information 2104. In some embodiments, image 2110 and visual features 2111 may be uploaded to apparatus 110 before a delivery route has begun, for example when delivery person 2001 collects the parcels for delivery. In some embodiments, image 2110 and visual features 2111 may be received and/or updated dynamically along the route, for example through network 240. Alternatively, or additionally, image 2110 and/or visual features 2111 may be received by computing device 120. Computing device 120 may then load image 2110 and/or visual features 2111 to apparatus 110 or store them for use in verifying parcel recipients. In some embodiments, image 2110, visual features 2111 and other information associated with profile 2100 may be stored in a temporary or dedicated storage location. This information may be removed after the delivery has been made, or before a subsequent delivery route.

FIG. 21B illustrates an example environment 2150 for delivery of a parcel consistent with the disclosed embodiments. Delivery person 2001 may be delivering a parcel 2151 intended for an individual at an address associated with environment 2150. Delivery person 2001 may deliver parcel 2151 to a recipient 2160 who may accept parcel 2151. In some embodiments, computing device 120 may be configured to display information pertaining to delivery of parcel 2151, including the delivery address (e.g., address 2102), the intended recipient's name 2101, a customer ID 2103, etc. In some embodiments, computing device 120 may display image 2110 showing the intended recipient. In order to verify that recipient 2160 is the individual intended to receive parcel 2151, apparatus 110 may capture an image of recipient 1260 using image sensor 220. The image, for example, may be similar to the image of environment 2150 depicted in FIG. 21B. In some embodiments, delivery person 2001 may initiate capture of the image, for example, using a button or other user input on apparatus 110. In some embodiments, delivery person 2001 may initiate the image capture through computing device 120, for example, through a mobile application. In other embodiments, the image capture may be automatic. For example, apparatus 110 may continuously capture images while delivery person 2001 is within environment 2150 and may select photos containing individual 2160 for analysis.

Processor 210 may be configured to process the image and may detect visual features 2161 of recipient 2160 from the image. As described above, visual features 2161 may include facial features of recipient 2160, such as the eyes, nose, cheekbones, jaw, or other physical features that may be used for identification (e.g., size, body shape, posture, clothing, nametags, etc.) Processor 210 may use one or more algorithms for analyzing the detected features, such as principal component analysis (e.g., using eigenfaces), linear discriminant analysis, elastic bunch graph matching (e.g., using Fisherface), Local Binary Patterns Histograms (LBPH), Scale Invariant Feature Transform (SIFT), Speed Up Robust Features (SURF), or the like.

To verify that recipient 2160 is the intended recipient, processor 210 may compare the captured image of recipient 2160 to image 2110 stored on apparatus 110. In some embodiments, this may include comparing visual features 2161 of recipient 2160 to visual features 2111 stored in apparatus 110. Apparatus 110 may be configured to use additional information from the image for verifying the parcel has been delivered correctly, such as address number 2153, which may be compared to address 2102 associated with the intended recipient in profile 2100. In some embodiments, more than one image of the individual may be used to verify recipient 2160. Further, more than one valid recipient may be associated with a delivery. For example, an intended recipient may designate a second individual, who may also be authorized to accept the parcel.

When recipient 2160 has been verified as the individual intended to receive parcel 2151, apparatus 110 may transmit an indication of the verification. In some embodiments, apparatus 110 may transmit the indication to server 250 through network 240. Based on the received indication, server 250 may mark the delivery as complete. In some embodiments, the indication may also be transmitted to computing device 120, either directly from apparatus 110, or from server 250. Computing device 120 may be configured to display a notification (e.g., on display 260) indicating to delivery person 2001 that the recipient 2160 has been verified. In some embodiments, an indication that the delivery has been completed may be transmitted to recipient 2160, for example, through client device 2010.

Apparatus 110 may further be configured to store a delivery proof based on the verification. For example, apparatus 110 may store the captured image of individual 2160. In some embodiments, the delivery proof may comprise the entire image captured by apparatus 110. Alternatively, the delivery proof may comprise a portion of the image including individual 2160. The delivery proof may include other information, such as identification information of parcel 2151, a time of delivery, a delivery address or location, etc. Additional information captured in the image may also be included in the delivery proof, such as an address number 2153, a label 2152 identifying parcel 2151 (e.g., by a barcode, tracking number, etc.) or various other information. The delivery proof may be stored locally on a memory of apparatus 110 (e.g., memory 550) and/or may be transmitted to computing device 120, server 250, and/or client device 2010 to be stored on those devices.

In some embodiments, processor 210 may be configured to determine a level of confidence associated with the verification of recipient 2160. The level of confidence may be based on the degree of match between visual features 2111 and visual features 2161. The level of confidence may be represented as a percentage (e.g., 0%-100% match), on a predefined scale (e.g., 1-10), through predefined confidence levels (e.g., low confidence, high confidence, etc.), or the like. In some embodiments, the confidence score may also be based on the amount or quality of information available to processor 210. For example, if recipient 2160 is far away and therefore a relatively low-resolution image is used, a lower confidence score may be assigned. In some embodiments, recipient 2160 may be verified by comparing the confidence score to a predetermined threshold (e.g., requiring a confidence score of at least 100%, 90%, 80%, 70% etc.).

Where recipient 2160 cannot be verified (or where the confidence score does not meet a threshold confidence level), apparatus 110 may generate an indication that recipient 2160 has not been verified. The indication may be transmitted to server 250 to indicate that apparatus 110 was unable to verify the recipient. In some embodiments, the indication may be transmitted to computing device 120, either from apparatus 110, or through server 250. Computing device 120 may be configured to display a notification (e.g., on display 260) indicating that recipient 2160 has not be verified. Accordingly, delivery person 2001 may perform a manual verification process according to traditional techniques. For example, delivery person 2001 may ask for a name of recipient 2160, request a signature of recipient 2160 (which may be entered through computing device 120, for example), request a photo ID card or other form of ID from recipient 2160, or the like. Delivery person 2001 may then manually confirm whether recipient 2160 has been verified through computing device 120 (e.g., through a mobile application, etc.). The delivery proof generated by apparatus 110 may still be stored in the event of a manual verification. For example, computing device 120 may receive the delivery proof (which may include a captured image of recipient 2160) and may store the delivery proof based on the manual verification. In other embodiments, computing device 120 may transmit an indication that individual 2160 has been manually verified to apparatus 110 and apparatus 110 may then store and/or transmit the delivery proof as described above.

In some embodiments, the verification process may be performed by a device other than apparatus 110. For example, in some embodiments, computing device 120 may perform the verification. In such embodiments, image 2110 and/or visual features 2111 may be stored on computing device 120, as described above. Apparatus 110 may capture an image of individual 2160 and may transmit the captured image to computing device 120, either through a direct connection (e.g., Bluetooth™, NFC, etc.) or through network 240. Computing device 120 may then verify whether recipient 2160 is the intended recipient. Computing device 120 may then transmit an indication to server 250 that recipient 1260 has been verified. Computing device 120 may further generate and store a delivery proof, which may contain an image of recipient 2160. The delivery proof may be stored locally on computing device 120 and/or may be stored on server 250. If individual 2160 cannot be verified, computing device 120 may display a notification for delivery person 2001 for performing a manual verification. Computing device 120 may further transmit an indication that individual 2160 could not be verified to server 250.

In some embodiments, the verification process may be performed by server 250. Accordingly, image 2110 and/or visual features 2111 may not be transmitted to apparatus 110 or computing device 120. Apparatus 110 may capture an image of recipient 2160 and may transmit the image to server 250 for verification. Apparatus 110 may detect and analyze visual features 2161 prior to transmitting the image, or server 250 may process the image to determine visual features 2161. Server 250 may then compare visual features 2161 to visual features 2111 to determine whether recipient 2160 is the individual intended to receive parcel 2151. Sever 250 may transmit an indication of whether recipient 210 has been verified to apparatus 110 and/or computing device 120.

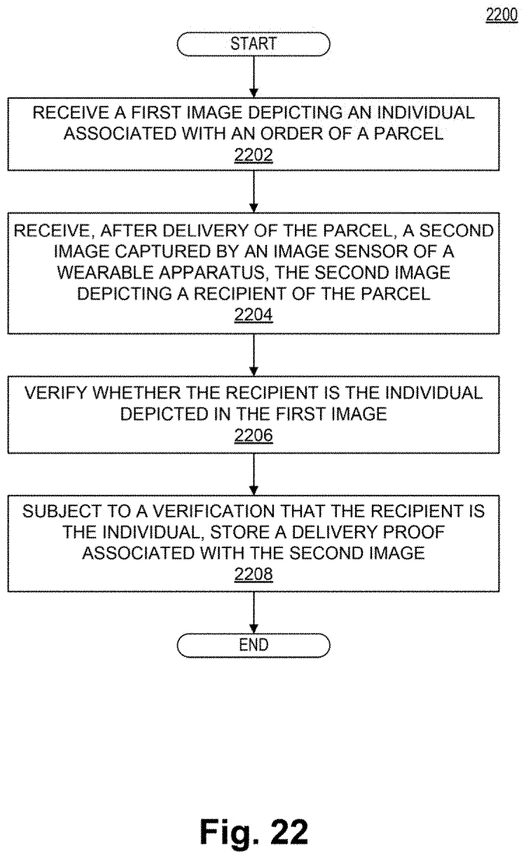

FIG. 22 is a flowchart showing an exemplary process 2200 for using a wearable apparatus for identification consistent with the disclosed embodiments. Process 2200 may be performed by at least one processing device, such as processor 210. Some or all of process 2200 may be performed by processors associated with components other than apparatus 110, such as computing device 120 (e.g., processor 540), server 250, and/or other devices.

At step 2202, process 2200 may include receiving a first image depicting an individual associated with an order of a parcel. For example, the first image may be image may be image 2110 described above. Accordingly, the first image may be stored in database 2051 and may be associated with an individual who is an intended recipient of a parcel. In some embodiments, the first image may be associated with an account of the individual. For example, the first image may be associated with an account of a delivery service, an account of a retailer, etc. The first image may be uploaded by the individual when placing an order for an item to be shipped to the individual. In some embodiments, the first image may be received from the individual (e.g., by a computing device associated with the individual). For example, the first image may be captured by a computing device associated with the individual, such as client device 2010. Alternatively, or additionally, the first image may be uploaded from storage by the individual, for example from client device 2010 or an external storage. In some embodiments, processor 210 may be programmed to transmit the second image for display on a computing device of the user, such as computing device 120.

After the parcel has been delivered (or before or during the delivery), at step 2204, process 2200 may include receiving, from an image sensor configured to capture a plurality of images from an environment of a user of a wearable apparatus, a second image captured by the image sensor, the second image depicting a recipient of the parcel. For example, delivery person 2001 may deliver the parcel to individual 2160, as described above. Apparatus 100, which may be worn by delivery person 2001, may capture the second image including individual 2160, using image sensor 220. In some embodiments, step 2204 may further comprise transmitting the second image for display on a computing device of the user, such as computing device 120.

At step 2206, process 2200 may include verifying whether the recipient is the individual depicted in the first image. For example, apparatus 110 may verify whether recipient 2160 is the individual depicted in image 2110. Accordingly, verifying whether the recipient is the individual depicted in the first image may comprise comparing the first image or features extracted therefrom to the second image or features extracted therefrom. In some embodiments, verifying whether the recipient is the individual depicted in the first image may comprise extracting features from the second image, such as features 2161, and comparing them with stored features associated with the individual, such as features 2111. Further, in some embodiments, the verification step may be performed by a processor other than processor 210 (e.g., by a processor of computing device 120 or server 250). Accordingly, in some embodiments, verifying from the second image whether the recipient is the individual depicted in the first image may comprise transmitting the second image or features extracted therefrom to a remote computing platform (e.g., server 250); and receiving, from the remote computing platform, an indication of whether the recipient is verified as the individual. The first image may similarly be transmitted to computing device 120 for verification, as discussed above.

At step 2208, process 2200 may include, subject to a verification that the recipient is the individual depicted in the first image, storing a delivery proof associated with the second image. In some embodiments, the delivery proof may comprise at least a portion of the second image. For example, the delivery proof may include a portion of the second image containing a representation of the recipient such that the delivery proof can be used to show that the recipient received the parcel. In some embodiments, the delivery proof may include other portions of the second image, which may include representations of the parcel, a label of the parcel (e.g., label 2152), a street or house number (e.g., address number 2153), etc. In some embodiments, storing the delivery proof may comprise storing the delivery proof on a local memory of the wearable apparatus, such as memory 550. Alternatively, or additionally, storing the delivery proof may comprise transmitting the delivery proof for storage on a remote storage device, such as server 250. The delivery proof may also be transmitted to and stored on computing device 120. In some embodiments, step 2208 may further comprise deleting the first and/or second image based on storing the delivery proof.

Process 2200 may include various other steps or substeps not shown in FIG. 22. For example, in some embodiments, process 2200 may further comprise, subject to a determination that the individual is not the recipient, providing an alert to the user. For example, providing the alert may comprise transmitting a notification to a computing device of the user. Based on the alert, delivery person 2001 may be prompted to perform a manual verification of the recipient, as discussed in greater detail above.

In some embodiments, recipient 210 may provide images of one or more additional individuals such as a family member, roommate, friend, concierge, or other individuals who are also authorized to receive the parcel for the recipient (e.g., if the recipient is not at home, etc.). In such embodiments, the features extracted from the captured image may be compared to features extracted from one or more of the stored images associated with the additional individuals. If there is a match with one of the stored images (either the intended recipient or the additional designated recipients), the identity may be confirmed as described above.

While the disclosed methods have been described with respect to delivery of a parcel, it is to be understood that process 2200 and the various embodiments discussed above may apply to other situations. For example, where the disclosed embodiments are used for admission to a facility, database 2051 may store profile information including images of individuals to be admitted to the facility. A user wearing apparatus 110, such as a bouncer or ticket taker, may capture an image of an individual attempting to access the facility. Apparatus 110 may compare the captured image to the image stored in database 2051 to determine if the person attempting to access the facility is the intended ticketholder. If the ticketholder is verified, apparatus 110 may store an admission proof, which may include the captured image. Process 2200 may similarly be applied to the other examples listed above, or any other process where an identify may be confirmed.

Pushing Images to a Wearable Apparatus

The disclosed systems and methods may enable a recognition system to recognize a person depicted in an image captured by a wearable apparatus based on a reference image of the person and identifying information received from an external device. For example, the user of the wearable apparatus may attend a conference where the user may meet many people. It may be helpful to recognize one or more of the people. Further, the user may or may not want to keep the images and names of the people once the conference is over. In such situations, wearable apparatus 110 may be configured to store images of the person the user encounters and identify persons based on the stored images. The images used to recognize the persons may be captured by wearable apparatus 110 or received from an external device (e.g., a server operated by the administrator of the conference). For example, the reference images of participants of the conference and identifying information associated with the participants may be pushed to wearable apparatus 110. Wearable apparatus 110 may capture images of the environment of the user and recognize one or more persons depicted in the captured images based on the reference images and the associated identifying information. Wearable apparatus 110 may also provide the user with the information of recognized persons by, for example, displaying the information to the user.

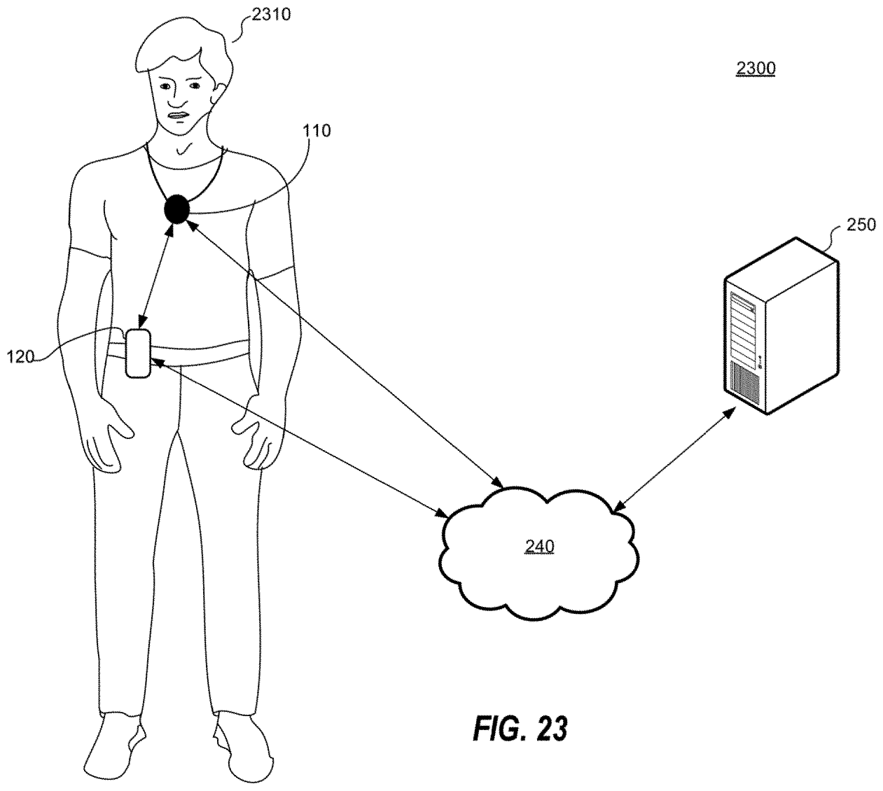

FIG. 23 illustrates an exemplary recognition system. Recognition system 2300 may include wearable apparatus 110, computing device 120, server 250, and network 240. User 2310 may wear wearable apparatus 110 as described elsewhere in this disclosure. Wearable apparatus 110 may be configured to capture one or more images of the environment of the user and recognize one or more persons and/or objects in the images. Computing device 120 and/or server 250 may provide additional functionality to wearable apparatus 110. For example, user 2310 may input a command into computing device 120 to receive a reference image depicting a person and identifying detail associated with the image from server 250, via, for example, network 240. Computing device 120 may send a request to server 250, which may transmit one or more reference images to wearable apparatus 110 and/or computing device 120 via network 240. Wearable apparatus 110 may use the reference image received to recognize the person depicted in the reference image. Network 240 may be configured to facilitate communications between the components of recognition system 2300.

Wearable apparatus may include at least one processor configured to cause wearable apparatus 110 to perform operations of wearable apparatus 110 described in this disclosure. Wearable apparatus 110 may be configured to capture one or more images of the environment of the user of wearable apparatus 110. For example, wearable apparatus 110 may include an image sensor configured to capture one or more images of the environment in the field-of-view of the user (or the image sensor).

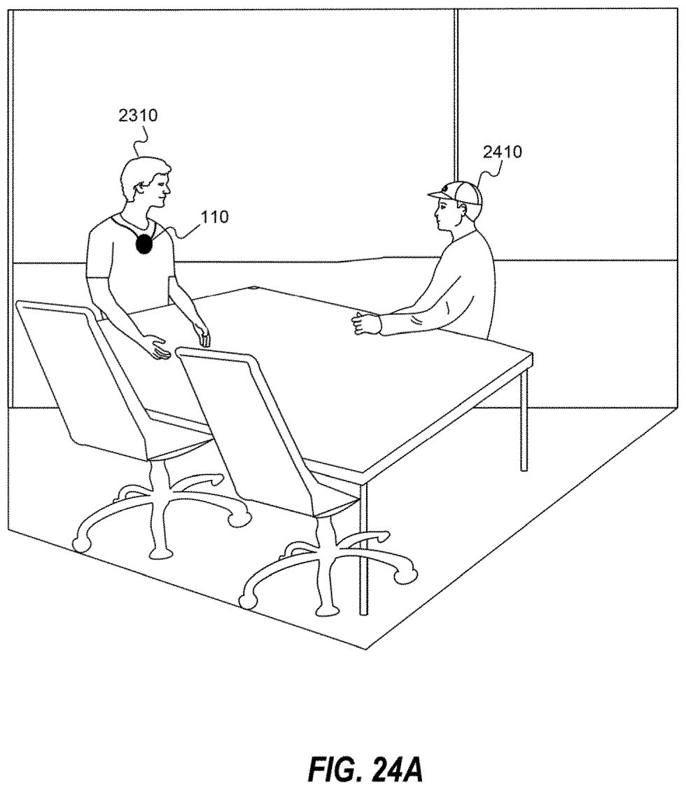

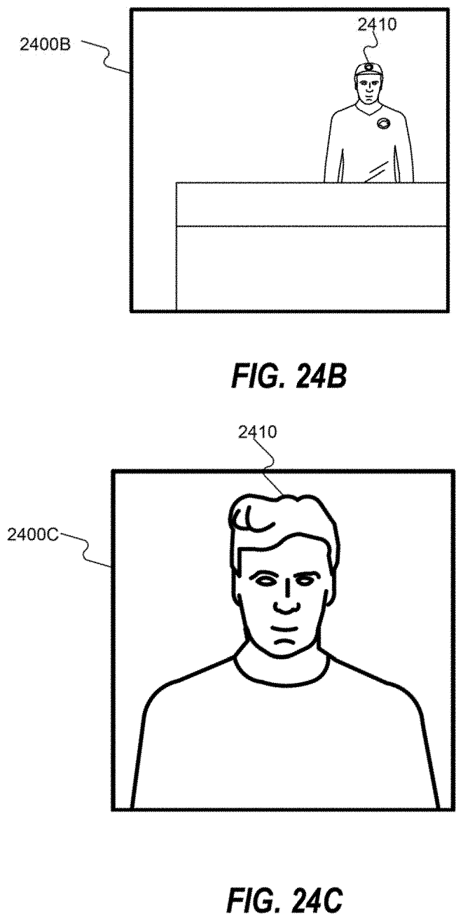

FIG. 24A is a schematic illustration of the environment of user 2310 wearing wearable apparatus 110. Wearable apparatus 110 may be configured to capture an image of the environment, such as image 2400B illustrated in FIG. 24B. Image 2400B may include a person 2410. In some embodiments, the image sensor may be configured to capture real-time image data of the environment.

Wearable apparatus 110 may also be configured to receive one or more reference images and identifying detail associated with the images from an external device (e.g., computing device 120, server 250, and/or a device of a third-party). For example, wearable apparatus 110 may be configured to receive a reference image 2400C illustrated in FIG. 24C from an external device. Image 2400C may depict person 2410. Wearable apparatus 110 may also receive identifying detail associated with image 2400C and/or person 2410. The term “identifying detail” refers to information identifying the person associated with a reference image. For example, wearable apparatus 110 may receive identifying detail such as personal information of person 2410, including, for example, the name, gender, age, profession, employer, title, or the like, or a combination thereof. Alternatively or additionally, identifying detail associated with a reference image may include characteristics of the person depicted in the reference image that can be used to recognize the person.

Wearable apparatus 110 may also be configured to store the received reference images and identifying detail associated with the images (or the persons) into a memory. Wearable apparatus 110 may further be configured to recognize one or more persons depicted in the image captured by the image sensor based on the reference images and the identifying detail associated with the reference images. In some embodiments, wearable apparatus 110 may be configured to display the results of the recognition to the user. For example, wearable apparatus 110 may include a display (or a display attached to wearable apparatus 110) configured to display the personal information of the person recognized in the images captured by the image sensor in real time. Alternatively or additionally, wearable apparatus 110 may transmit the recognition results to computing device 120 for display.

Computing device 120 may be configured to communicate with wearable apparatus 110 and assist wearable apparatus 110 to perform the operation thereof. For example, when the user arrives at a conference, the user may input a command at computing device 120 to scan a code for receiving one or more reference images. Computing device 120 may be configured to scan the code, and one or more reference images may be transmitted (or pushed) to computing device 120 and/or wearable apparatus 110 by an external device (e.g., server 250). In some embodiments, computing device 120 may be configured to control wearable apparatus 110 to perform various operations. For example, computing device 120 may receive user input to delete one or more images and associated with information stored in wearable apparatus 110.

In some embodiments, computing device 120 may include, for example, a smartphone, or a tablet, or a dedicated processing unit, which may be portable (e.g., can be carried in a pocket of user 100). Although shown in FIG. 23 as an external device, in some embodiments, computing device 120 may be provided as part of wearable apparatus 110, configured to perform one or more operations of wearable apparatus 110 described in this disclosure. For example, computing device 120 may be configured to receive images from wearable apparatus 110 and recognize one or more persons in the images captured by wearable apparatus 110.

Server 250 may be configured to store one or more reference images and identifying detail associated with the reference images. Server 250 may also be configured to transmit or push one or more reference images and the associated identifying detail to wearable apparatus 110 and/or computing device 120. In some embodiments, server 250 may be operated by a third party.

Wearable apparatus 110 may be configured to communicate with computing device 120 and server 250 via any known wireless standard (e.g., Wi Fi, Bluetooth®, etc.), as well as near-field capacitive coupling, and other short-range wireless techniques, or via a wired connection. Alternatively or additionally, wearable apparatus 110 may be configured to communicate with computing device 120 and server 250 via network 240. Alternatively or additionally, wearable apparatus 110 may be configured to communicate with a device of a third-party via network 240. For example, wearable apparatus 110 may be configured to receive one or more images and identifying detail associated with the images from a device of a conference host.

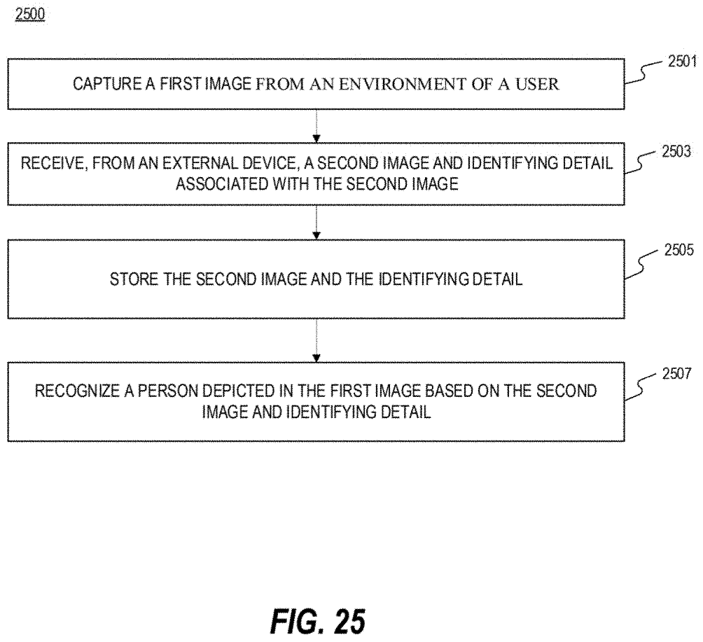

FIG. 25 illustrates a flowchart of an exemplary process 2500 for recognizing a person depicted in an image captured by wearable apparatus 110. In some embodiments, one or more steps of process 2500 may be performed by at least one processor of wearable apparatus 110.

At step 2501, wearable apparatus 110 may be configured to capture a first image from an environment of a user of the wearable apparatus. For example, wearable apparatus 110 may include an image sensor configured to capture an image from the environment of the user. The image sensor may transmit the image data of the image to at least one processor of the wearable apparatus 110 for processing. As discussed earlier, FIG. 24A is a schematic illustration of an environment of user 2310 wearing wearable apparatus 110. Wearable apparatus 110 may be configured to capture an image of the environment, such as image 2400B illustrated in FIG. 24B. Image 2400B may include a person 2410. In some embodiments, the image sensor may be configured to capture real-time image data of the environment.

At step 2503, wearable apparatus 110 may be configured to receive, from an external device, a second image and an identifying detail associated with the second image. For example, wearable apparatus 110 may receive a reference image and an identifying detail from server 250 via, for example, network 240. In some embodiments, a handshake protocol may be applied between wearable apparatus 110 and the external device to ensure that the images and associated identifying details are received from a safe source. By way of example, wearable apparatus 110 may be configured to receive a reference image 2400C illustrated in FIG. 24C from server 250. Image 2400C may depict person 2410. Wearable apparatus 110 may also receive identifying detail associated with image 2400C and/or person 2410. The identifying detail associated with a reference image and/or the person depicted in the reference image may be used to recognize the person in other images (e.g., an image captured by wearable apparatus 110). For example, the identifying detail may include the personal information of the person depicted in the reference image.

In some embodiments, the external device may send or push one or more second (or reference) images and associated identifying detail to wearable apparatus 110 in response to an event trigger. For example, the external device may determine the position of wearable apparatus 110 based on GPS information associated with the user, which may indicate that the user walks into a conference. The external device may push one or more second images and associated identifying details to wearable apparatus 110 (and/or a device associated with the user) based on the position of the user. As another example, in a conference, each participant may grant a privilege to an administrator of the conference to push images to the participant's wearable apparatus. When the user arrives at the conference, the user's image may be taken and transmitted to the external device. For example, the user's image may be taken by wearable apparatus 110 and transmitted to the external device. Alternatively, the external device may already have a reference image of the user in a storage. The external device may push the user's reference image and associated identifying detail to other participants, and push reference images of other participants and associated with identifying details to the user's wearable apparatus 110 (or computing device 120). In some embodiments, all reference images and identifying detail may be stored and pushed to the device of any newly arriving participant by the external device. As another example, when a new employee (e.g., the user of wearable apparatus 110) joins an organization, the employee's image (i.e., the reference image) may be taken and pushed to the devices of other employees, and the reference images of one or more of the employees may be pushed to wearable apparatus 110. As still another example, when a patient is admitted to a hospital or a clinic, the patient's image may be captured, and his or her identifying detail (e.g., the personal information) and image may be pushed to the wearable apparatuses of the personnel members of the hospital. Thus, when a personnel member meets the patient, the wearable apparatus of the personal member may recognize the patient as described elsewhere in this disclosure. In some embodiments, a link to the patient's medical records may also be associated with the reference image and identifying detail, such that the records can be accessed by the personnel member.

In some embodiments, the external device may transmit or push one or more reference images and associated identifying details to wearable apparatus 110 based on an identification sharing policy. For example, the identification sharing policy may specify the recipient(s) of one or more images and associated identifying details, and the external device may determine whether the user of wearable apparatus 110 is authorized to receive one or more images and identifying details. By way of example, the external device may push a reference image of a patient and associated identifying detail to devices of all personnel members of the hospital based on the identification sharing policy. Alternatively, the external device may determine that a subset of personnel members are authorized to receive a reference image and identifying details based on the identification sharing policy. The external device may also push the reference image and associated identifying detail to these relevant members, such as personnel members of the particular unit the patient is admitted to. In some embodiments, the medical records of the patient may be made available only to personnel members with adequate permissions (e.g., as described in the identification sharing policy), to ensure patient confidentiality. Selective push may reduce the energy consumption, and the number of false alarms, as well as the number of true but unrequired recognition which will result in unnecessarily bothering the personnel member.

In some embodiments, wearable apparatus 110 may be configured to receive one or more reference images and associated identifying detail from an external device in response to a command sent from a device associated with the user (e.g., computing device 120). For example, computing device 120 may receive input from the user to receive one or more reference images and transmit a command to wearable apparatus 110, which may receive one or more reference images from an external device in response to the command received. Alternatively or additionally, computing device 120 may transmit a request to receive reference images to the external device, which may push reference images to wearable apparatus 110 in response to the request.

In some embodiments, computing device 120 may scan a code, and wearable apparatus 110 may receive one or more reference images in response to the scan of the code. For example, computing device 120 may be prompted to scan a code, such as a quick response (QR) code, which may cause computing device 120 to, for example, activate an application to access one or more reference images and associated identifying details. Wearable apparatus 110 may also receive one or more reference images and associated identifying details from the external device. In some embodiments, the access of reference images and associated identifying details may be subject to another condition, such as entering a password provided to the user, a location as received from a GPS or through registering with a local network, or the like, in order to prevent unwanted users from accessing the information.

In some embodiments, wearable apparatus 110 may receive one or more reference images and associated identifying details from computing device 120. Alternatively or additionally, wearable apparatus 110 may receive one or more reference images and associated identifying details from a storage of wearable apparatus. For example, wearable apparatus 110 may store one or more reference images and associated identifying details received previously (e.g., relating to a conference of the last year) and obtain the reference images and associated identifying details when needed. In some embodiments, wearable apparatus 110 may store one or more images captured by wearable apparatus 110 as reference images along with associated identifying details provided by the user. For example, wearable apparatus 110 may capture an image depicting a person who the user recently met, and the user may input the identifying detail associated with the image and/or the person. Wearable apparatus 110 may also be configured to save the image as a reference image of the person and the associated identifying detail into a storage.

At step 2505, wearable apparatus 110 may be configured to store the second image and the identifying detail in association with the second image. For example, wearable apparatus 110 may store the reference image(s) and associated identifying detail in a storage of wearable apparatus 110. Alternatively or additionally, one or more reference images and associated identifying details may be saved into a storage of computing device 120, which may be accessed by wearable apparatus 110 if needed.

At step 2507, wearable apparatus 110 may be configured to recognize a person depicted in the first image (captured by wearable apparatus 110) based on the second image (received from the external device) and the identifying detail associated with the second image. For example, wearable apparatus 110 may be configured to capture a first image from the environment of the user in real time and recognize the person depicted in the first image based on a reference image depicting the same person and associated identifying detail received from the external device. Wearable apparatus 110 may use the reference images received from the external device to recognize the person so that the recognition process may be limited to a small or subset set of images (e.g., the reference images received from the external device) and associated identifying details. In doing so, wearable apparatus 110 may limit the search for a match for the person depicted in the image it captured among the predetermined set of reference images, which may reduce computation requirements for the recognition.

In some embodiments, wearable apparatus 110 may use a deep learning algorithm to recognize a person depicted in the first image based on one or more reference images received from an external device.

In some embodiments, wearable apparatus 110 may also provide the results of the recognition to the user via wearable apparatus 110 and/or computing device 120. The results of the recognition may include personal information, such as the name and title, of the recognized person. For example, wearable apparatus 110 may include a display configured to present the identification information (e.g., the name) of the person to the user. Alternatively or additionally, wearable apparatus 110 may transmit the results of the recognition to glasses 130 and/or computing device 120 to present the identification information of the person to the user. Alternatively or additionally, wearable apparatus 110 may include a speaker configured to provide the identification information of the recognized person in form of audio to the user.

In some embodiments, wearable apparatus 110 may be configured to provide an indication that the second image is received from an external device upon recognition of the person. For example, wearable apparatus 110 may provide an indication to the user that the recognized person belongs to the group of people whose image was pushed to wearable apparatus 110.

In some embodiments, wearable apparatus 110 may be configured to delete one or more reference images and associated identifying details. For example, wearable apparatus 110 may delete one or more reference images and associated identifying details in response to user input from the user. Alternatively or additionally, wearable apparatus 110 may delete one or more reference images and associated identifying details in response to a command sent from computing device 120 and/or the external device. For example, computing device 120 may receive input from the user to delete one or more reference images and transmit a command to wearable apparatus 110 to delete the reference image(s) and associated identifying detail. Wearable apparatus 110 may be configured to delete the reference image(s) and associated identifying detail based on the command.

In some embodiments, wearable apparatus 110 may be configured to delete one or more reference images and associated identifying details in response to a command sent from the external device to wearable apparatus 110 and/or computing device 120. For example, the external device may send a command to wearable apparatus 110 and/or computing device 120 to delete one or more reference images and associated identifying details based on an event trigger. By way of example, the external device may determine the position of wearable apparatus 110 based on GPS information associated with the user, which may indicate that the user walks out of a conference. The external device may transmit a delete command to wearable apparatus 110, which may delete the reference images and associated identifying detail specified in the command. In some embodiments, the external device may also transmit a delete command to the devices of the other participant to delete the reference image of the user of wearable apparatus 110. As another example, wearable apparatus 110 may receive an indication that the conference is over, and in response to the indication, wearable apparatus 110 may delete one or more reference images and associated identifying details. Alternatively, wearable apparatus may delete one or more reference images and associated identifying details in a predetermined period of time (e.g., three days) after the conference is over.

As another example, when an employee (e.g., the user of wearable apparatus 110) leaves an organization, the external device may transmit a command to wearable apparatus 110 to delete the reference images and associated identifying detail relating to the organization. In some embodiments, the external device may also transmit a command to other devices to delete the reference image and associated identifying detail of the user. As still another example, when a patient is discharged from the hospital, the external device may transmit a command to devices of personnel members to delete the reference image and associated identifying detail of the patient.

In some embodiments, wearable apparatus 110 may be configured to delete one or more reference images and associated identifying details based on a predetermined period of time. For example, wearable apparatus 110 may delete one or more reference images and associated identifying details in three days of receiving the reference images from the external device.

In some embodiments, wearable apparatus 110 may be configured to determine (or receive an indication indicating) that the user has not seen or talked to (or spent time with) a person depicted in one or more reference images for a predetermined period of time or until an event trigger, such as the end of the conference. Wearable apparatus 110 may delete the reference image and identifying detail associated with the person based on the determination (or indication). Alternatively or additionally, wearable apparatus 110 may be configured to determine (or receive an indication indicating) that the user has seen or talked to (or spent time with) a person depicted in one or more reference images within a predetermined period of time. Wearable apparatus 110 may not delete the reference image and identifying detail associated with the person based on the determination or indication (e.g., by forgoing an action of deleting the reference image and identifying detail).

In some embodiments, wearable apparatus 110 may receive an indication that one or more reference images and associated identifying details are not to be deleted. For example, wearable apparatus 110 may receive user input from the user not to delete a reference image of a person and the associated identifying detail. In some embodiments, wearable apparatus 110 may be configured to selectively save one or more reference images and associated identifying details for future use. For example, wearable apparatus 110 may receive user input from the user to save a reference image and associated identifying detail for recognizing the person in the future. Wearable apparatus 110 may tag the reference image as not to be deleted and may not delete the reference image despite wearable apparatus 110 may receive a delete command.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments. Additionally, although aspects of the disclosed embodiments are described as being stored in memory, one skilled in the art will appreciate that these aspects can also be stored on other types of computer readable media, such as secondary storage devices, for example, hard disks or CD ROM, or other forms of RAM or ROM, USB media, DVD, Blu-ray, Ultra HD Blu-ray, or other optical drive media.

Computer programs based on the written description and disclosed methods are within the skill of an experienced developer. The various programs or program modules can be created using any of the techniques known to one skilled in the art or can be designed in connection with existing software. For example, program sections or program modules can be designed in or by means of .Net Framework, .Net Compact Framework (and related languages, such as Visual Basic, C, etc.), Java, C++, Objective-C, HTML, HTML/AJAX combinations, XML, or HTML with included Java applets.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

1. A wearable apparatus, the wearable apparatus comprising:
an image sensor configured to capture a plurality of images from the environment of a user of the wearable apparatus;
an audio sensor configured to capture sound from the environment of the user; and
at least one processor programmed to:
receive the plurality of images captured by the image sensor;
receive an audio signal representative of the sound captured by the audio sensor;
determine, based on at least one of the plurality of images or the audio signal, whether an individual within the environment of the user is a recognized individual of the user;
subject to a determination the individual is not a recognized individual, identify the individual based on an external resource;
identify a content source associated with the individual;
identify a first content item associated with the individual; and
provide the first content item to a computing device associated with the user.

2. The wearable apparatus of claim 1, wherein the at least one processor is further programmed to:
retrieve, subject to a determination that the individual is a recognized individual, a second content item associated with a previous encounter between the user and the individual; and
provide the second content item to the computing device associated with the user.

3. The wearable apparatus of claim 2, wherein the second content item comprises at least one of a name or a vocal pronunciation of a name of the individual.

4. The wearable apparatus of claim 1, wherein identifying the individual based on the external resource comprises performing an image search based on a representation of the individual depicted in the plurality of images.

5. The wearable apparatus of claim 1, wherein the computing device is configured to display the first content item to the user.

6. The wearable apparatus of claim 1, wherein the content source comprises a social network platform, and the first content item comprises one or more posts associated with the individual on the social network platform.

7. The wearable apparatus of claim 1, wherein the content source comprises a blog, and the first content item comprises one or more posts associated with the individual on the blog.

8. The wearable apparatus of claim 2, wherein the second content item is retrieved from a memory of the wearable apparatus.

9. The wearable apparatus of claim 2, wherein the second content item is retrieved from a network storage location.

10. The wearable apparatus of claim 2, wherein the previous encounter comprises a previous conversation between the user and the individual.

11. The wearable apparatus of claim 10, wherein the second content item comprises a topic of conversation associated with the previous conversation.

12. The wearable apparatus of claim 10, wherein the second content item comprises at least a partial transcript of the previous conversation.

13. A method for using a wearable apparatus in social events, the method comprising:
receiving a plurality of images captured from an environment of a user of a wearable apparatus by an image sensor;
receiving an audio signal representative of a sound captured from the environment of the user by an audio sensor;
determining, based on at least one of the plurality of images or the audio signal, whether an individual within the environment of the user is a recognized individual of the user;
subject to a determination the individual is not a recognized individual, identifying the individual based on an external resource;
identifying a content source associated with the individual;
identifying a first content item associated with the individual; and
providing the first content item to a computing device associated with the user.

14. The method of claim 1, wherein the method further comprises:
subject to a determination that the individual is a recognized individual, retrieving a second content item associated with a previous encounter between the user and the individual; and
providing the second content item to the computing device associated with the user.

15. The method of claim 14, wherein the second content item comprises at least one of a name or a vocal pronunciation of a name of the individual.

16. The method of claim 12, wherein identifying the individual based on the external resource comprises performing an image search based on a representation of the individual depicted in the plurality of images.

17. The method of claim 12, wherein the computing device is configured to display the first content item to the user.

18. The method of claim 12, wherein the content source comprises a social network platform, and the first content item comprises one or more posts associated with the individual on the social network platform.

19. The method of claim 12, wherein the content source comprises a blog, and the first content item comprises one or more posts associated with the individual on the blog.

20. The method of claim 14, wherein the second content item is retrieved from a memory of the wearable apparatus.

21. The method of claim 14, wherein the second content item is retrieved from a network storage location.

22. The method of claim 14, wherein the previous encounter comprises a previous conversation between the user and the individual.

23. The method of claim 22, wherein the second content item comprises a topic of conversation associated with the previous conversation.

24. The method of claim 22, wherein the second content item comprises at least a partial transcript of the previous conversation.

25.-67. (canceled)
